(19) United States
(12) Patent Application Publication
(10) Pub. No.: US 2011/0142986 A1
(43) Pub. Date: Jun. 16, 2011

(54) TRANSCONJUGANTS OF LACTIC ACID BACTERIA

(75) Inventors: Jakob Brandt Borup Haaber, Copenhagen N (DK); Karin Hammer, Rungsted Kyst (DK); Sylvain Moineau, Quebec (CA)

(73) Assignee: DANMARKS TEKNISKE UNIVERSITET, Lyngby (DK)

(21) Appl. No.: 13/000,972

(22) PCT Filed: Jul. 2, 2009
(86) PCT No.: PCT/DK2009/050157

§ 371 Date: Feb. 16, 2011

(30) Foreign Application Priority Data
Jul. 2, 2008 (DK) PCT/DK2008/050166
Oct. 31, 2008 (DK) PA 2008 01510

(51) Int. Cl. A23L 1/00; C12N 1/20; C12N 15/74

(52) U.S. Cl. 426/7; 435/252.3; 426/61; 435/471

(57) ABSTRACT

The present invention relates to the field of dairy science. In particular, the present invention relates to methods for improving dairy starter culture quality as well as food products that can be obtained using such methods.

### TECHNICAL FIELD OF THE INVENTION

The present invention relates to the field of dairy science. In particular the present invention relates to methods for improving dairy starter culture quality. The present invention further relates to transconjugants of lactic acid bacteria such as Lactococcus, Streptococcus, Lactobacillus, Leuconostoc, Pediococcus and methods of preparing the same.

### BACKGROUND OF THE INVENTION

Lactic acid bacteria such as Lactococcus lactis are used in milk fermentations world wide in the dairy industry to produce a variety of cultured dairy products. Phage infections can ruin the fermentation by inactivating the inoculated cultures.

Phages are the major cause of fermentation failures during the manufacture of these cultured dairy products. There is thus a permanent need in the art for L. lactis starter cultures to perform at a high level of consistency and efficiency.

### Phages

Lactococcal phages are characterized by having relatively short latent periods and relatively large burst sizes. They are the major cause of fermentation failure leading to production loss in the dairy industry. Lactococcal phages are currently divided into eight distinct groups of which three groups namely “936”, “c2” and “P335” are responsible for the vast majority of phage attacks in industrial fermentations. The genomes of the phages within one single group are highly conserved except for the P335 group.

Industrial fermentations are carried out in large fermentation vats in a non-sterile environment. Prior to fermentation, the ingredients are usually pasteurized. However, the phages are often resistant to the pasteurization process. Presence of phages can lead to variations in flavor and texture of the fermented dairy product or even loss of the entire production with serious economical loss as a consequence. The dairy industry is therefore using a variety of methods in limiting phage attacks. Such approaches include e.g. improved disinfection processes, rotation of starter cultures and application of phage resistant starter strains.

### Phage Defense Mechanisms

During evolution lactic acid bacteria such as L. lactis has developed a series of defense mechanisms against phage attacks. These naturally occurring phage resistance mechanisms (φrm) has been studied extensively and also applied in industrial starter cultures. Most of the naturally occurring φrms are found on plasmids and they are classified into four groups according to their mode of action: 1) adsorption inhibition, 2) blocking of phage DNA injection, 3) restriction/modification systems (R/M) and 4) abortive infection mechanisms (Abi). Among these defense mechanisms, the Abi systems are considered to be the most powerful due to their diverse mode of action and efficiency against the most common phages.

### Abi Mechanisms

Abi mechanisms function in the phage life cycle subsequent to the injection of phage DNA into the bacterial cell—typically after expression of early phage genes. As a consequence, the phage lytic cycle is terminated and usually the host dies. Very few viable phage progeny are thus released and the phenotypic outcome is a reduction in the number and size of plaques and thus a reduction of the severity of the phage infection.

To date, twenty-two lactococcal Abi systems have been isolated. These Abi systems target one, two or all three groups of the common phage species 936, c2, P335 with varying efficiency (EOP values from 10⁻¹ to <10⁻⁸).

Most of the isolated Abi systems are found on plasmids of which many are conjugative. By sharing the plasmid encoded φrms within the bacterial population, conjugation thus provides an adaptation strategy to the phage containing dairy environment.

The point of interference with the phage life cycle has been determined to some degree for most of the Abi mechanisms:
AbiA, AbiF, AbiK, AbiP, AbiR, and AbiT apparently interfere with phage DNA replication.
AbiC apparently interfere with capsid production.
AbiE, AbiI, and AbiQ apparently interfere with phage packaging.
AbiB is apparently an RNase.
AbiD1 seems to interfere with a phage RuvC-like endonuclease.
AbiU apparently delays phage transcription.
AbiZ apparently causes premature lysis of the infected cell.

Though the point of action in the phage life cycle has been determined, the phage protein interacting with the Abi mechanism has only been identified in AbiA, AbiD1, AbiK and AbiP. An increasing number of phage genomes are being sequenced providing a bulk of sequence data in which numerous putative proteins are found. However, experimental evidence for the function of these proteins are lacking behind.

Several phage resistant strains of L. lactis have been constructed by introducing Abi systems in phage sensitive industrial starter cultures. However, extensive use of these bacterial cultures leads to problems with emergence of phage mutants capable of overcoming the introduced abi systems.

The evolutionary “arms race” between phage mutants and bacterial φrms means that there is a constant need in the art for identifying novel natural φrms. There is a particular need in the art for finding novel Abi-mechanisms that interact with previously unknown targets in the phage. Furthermore there is a need in the art for novel Abi-mechanisms in Lactococcus bacteria that do not classify as GMO.

Finally there is a need in the art for identifying φrms that provide efficient protection against phages.

### SUMMARY OF THE INVENTION

One aspect of the present invention relates to a transconjugated lactic acid bacterium, wherein the transconjugated genetic material is chromosomal DNA encoding a genetic determinant for a phage resistance mechanism (φrm).

The present invention further relates to Lactococcus transconjugants comprising a chromosomal encoded phage resistance mechanism. In particular, Lactococcus transconjugants, wherein the transconjugated genetic material is a chromosomal encoded phage resistance mechanism

Another aspect concerns a starter culture composition comprising a Lactococcus transconjugant of the invention. Yet another aspect concerns method for preparing a fermented food product, said method comprising adding to the raw material or semi-manufacture to be fermented at least one of the components selected from the list consisting of: a Lactococcus transconjugant according of the invention, and a starter culture according of the invention. A further aspect relates to a food product comprising a transconjugant of the invention such as a transconjugant Lactococcus. One aspect of the invention relates to use of a transconjugant lactic acid bacterium of the invention (such as a Lactococcus transconjugant) in the production of a food product.

One particular aspect of the invention relates to a method of preparing a transconjugated lactic acid bacterium according to any of the preceding claims, said method comprising culturing a donor bacterium comprising chromosomal DNA encoded said genetic determinant for said phage resistance mechanism and a recipient lactic acid bacterium in a medium suitable for transconjugation, and subsequently isolating a transconjugated lactic acid bacterium conferring said phage resistance. In one embodiment said recipient lactic acid bacterium is a Lactococcus bacterium

A further aspect concerns the use of bacterium comprising a chromosomal encoded phage resistance mechanism as donor for the preparation of a transconjugant bacterium comprising a chromosomal encoded phage resistance mechanism. A final aspect relates to a transconjugated lactic acid bacterium obtained by a method according to the above method. In one embodiment said transconjugated lactic acid bacterium is a Lactococcus transconjugant.

The present invention also relates to methods for producing a fermented dairy product as well as the products resulting from these processes.

The novel transconjugant according to the present invention provide a number of advantages as described in the following.

### BRIEF DESCRIPTION OF THE FIGURES

FIG. 1: Characteristics of the twenty-two (including AbiV from the present invention) Abi mechanisms isolated to date.

FIG. 2: The sequence from GenBank (acc.nr AF324839) containing orf1 which surprisingly turned out to have the capability to function as a φrm according to the present invention. FIG. 2A: the strains with the transposon containing vector pGhost9::ISS1 inserted on the chromosome. Arrows indicate the position and direction of the inserted ISS1 sequences. The presence of a promoter and the φrm⁺ phenotype is indicated to the right. FIG. 2B: the strains with the cloned fragment including orf1. The lines represent the cloned DNA fragment, and the x in JH-24 represent the position of the frame shift mutation introduced into this strain.

FIG. 3: Time course experiment of a phage infection. Samples are taken during infection of phage resistant Lactococcus lactis strain JH-20 (upper panel) and phage sensitive Lactococcus lactis strain JH-16 (lower panel) with p2 phage. The experiment was run for 120 min and samples were taken at: −10, 0, 10, 20, 30, 40, 50, 60, 90 and 120 minutes. Total DNA was isolated from the cells and restricted with EcoRI. The resulting restriction fragments are representing EcoRI digested p2 DNA. Band 1.3 kb and 4 kb are spanning the cos site which marks the extremities of the phage DNA. The cos site is cut during packaging of phage DNA in the lytic life cycle of the wt phage, revealing mature phage DNA molecules in units of one genome. In the phage resistant Abi mutant, the cos site is not cut resulting in non-mature phage DNA that can not be packed into the phage capsids. The figure thus shows that production of mature phage DNA is significantly decreased in the strains containing the AbiV mechanism.

FIG. 4: DNA sequence of the 1.3 kb DNA fragment (bp 1021-2320 in GenBank acc.nr AF324839) cloned in vector pJH2. This fragment comprises orf1 (bp 1276-1878) encoding the φrm. Ribosome binding site is underlined in nucleotides matching the lactococcal consensus sequence (AGAAAGGAGGT). The translated amino acids are shown below the DNA sequence.

FIG. 5: DNA sequence of the 499 by DNA fragment from phage p2 containing orf26 and the upstream region towards orf27. Ribosome binding site is underlined in nucleotides matching the lactococcal consensus sequence (AGAAAGGAGGT). The translated amino acids are shown below the DNA sequence.

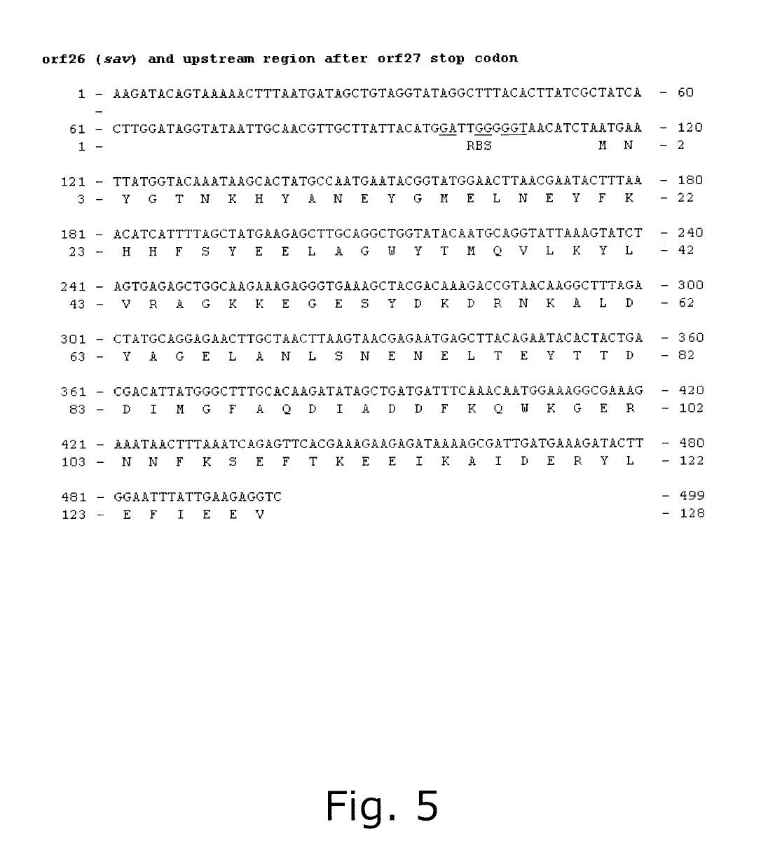

FIG. 6: Reverse transcriptase PCR carried out on isolated RNA. (A) Experiment done with reverse transcriptase enzyme. (B) Control without reverse transrciptase. Lanes 1-4 represents: JH-80 (spontaneous mutant), JH-20 (high expression of AbiV), JH-54 (wt), JH-32 (insertional mutant), respectively. L is Generuler ladder (Fermentas).

FIG. 7: RT-PCR assays carried out on RNA isolated from various L. lactis strains. Panel A. Expression of AbiV. Experiments performed in presence of reverse transcriptase. Panel B. As in panel A. However, experiments performed in the absence of reverse transcriptase. Panel C. Expression of Trans. Experiments performed in presence of reverse transcriptase. Lane 1, L. lactis JH-80 (spontaneous BIM); Lane 2, L. lactis JH-32 (insertional mutant expressing abiV); Lane 3, L. lactis JH-20 (AbiV cloned into an expression vector); Lane 4, L. lactis JH-54 (empty vector); Lane 5, positive PCR control using genomic DNA from L. lactis MG1363; L, Generuler ladder (Fermentas).

The present invention will now be described in more detail in the following.

### DETAILED DESCRIPTION OF THE INVENTION

### Definitions

Prior to discussing the present invention in further details, the following terms and conventions will first be defined:

### Phage

A bacteriophage (from ‘bacteria’ and Greek phagein, ‘to eat’) is any one of a number of virus-like agents that infect bacteria. The term is commonly used in its shortened form, phage. Typically, bacteriophages consist of an outer protein shell (called capsid or head) enclosing genetic material. The genetic material can be ssRNA, dsRNA, ssDNA, or dsDNA between 5 and 500 kilo base pairs long with either circular or linear arrangement. Bacteriophages are much smaller than the bacteria they destroy—usually between 20 and 200 nm in size. Phages according to the present invention have the ability to infect lactic acid bacteria such as bacteria of the genus Lactococcus.

### Phage Resistance Mechanism (φrm)

A functional phage resistance mechanism is herein meant to be a mechanism (a gene product(s) which is either a polynucleotide(s) or a polypeptide(s)) that directly inhibits the phage lytic life cycle. They are classified into four groups according to their mode of action: 1) interference with phage adsorption (adsorption inhibition), 2) blocking of phage DNA injection, 3) restriction/modification systems (R/M) and 4) abortive infection mechanisms (Abi). Although Abi mechanisms are the preferred φrms due to their diverse mode of actions, the present invention is not limited to Abi mechanisms. In one embodiment, the φrm is a restriction/modification system (R/M) or a phage resistance mechanism that inhibit adsorption or blocking of phage DNA injection.

In a particular embodiment, phage resistance mechanisms as used herein furthermore denote mechanisms that work in synergy with a phage encoded product. As an example hereof, the present invention relates to use of SEQ ID NO 1 for conferring phage resistance to bacterial cells as well as the use of SEQ ID NO 1 in combination with SEQ ID NO 2 or SEQ ID NO 7 for obtaining an even more efficient phage resistance mechanism than was possible when only using SEQ ID NO 1.

### Abi—Abortive Infection Mechanisms

Abortive infection system (Abi) refers to a phage resistance system other than the restriction/modification systems (R/M), which prevents phage proliferation after the phage DNA has entered the host cell. The point of interference with the phage life cycle has been determined to some degree for most of the Abi systems:
AbiA, AbiF, AbiK, AbiP, AbiR, and AbiT apparently interfere with phage DNA replication.
AbiC apparently interfere with capsid production.
AbiE, Abil, and AbiQ apparently interfere with phage packaging.
AbiB is apparently an RNase.
AbiD1 seems to interfere with a phage RuvC-like endonuclease.
AbiU apparently delays phage transcription.
AbiZ apparently causes premature lysis of the infected cell.

Abi refers to the genetic determinant for the abortive infection mechanism such as AbiA, AbiF, AbiK, AbiP, AbiR, AbiT, AbiC, AbiE, Abil, AbiQ, AbiB, AbiD1, AbiU, AbiZ and AbiV. Accordingly, the bacterium comprising an activated Abi genetic determinant adopts the phage resistance phenotype corresponding to said Abi.

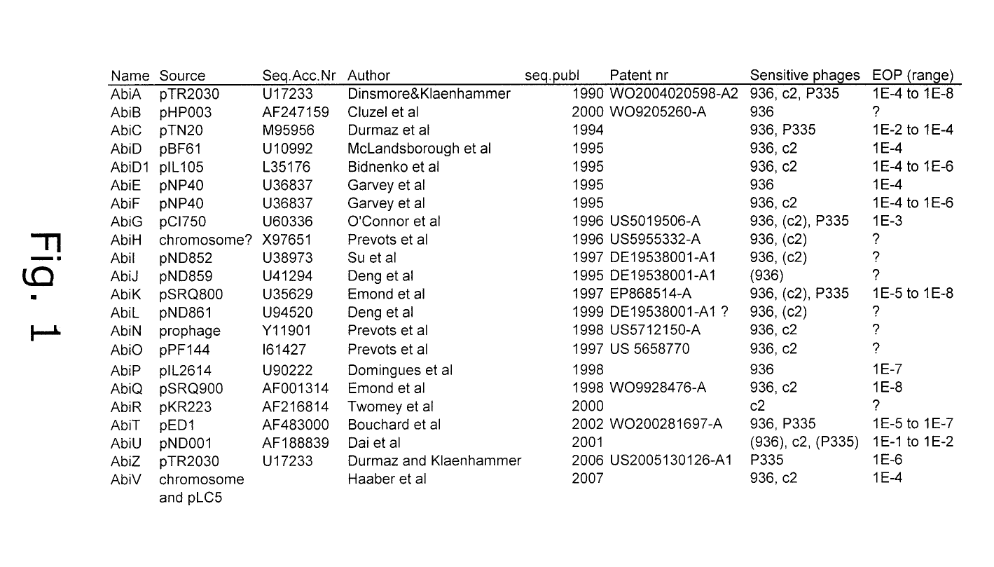

Abi locus refers to a locus comprising a least one Abi gene encoding a gene product, which upon expression confer the Abi mechanism to the host cell identified by the Abi phenotype. The Abi gene product involved in or responsible for the Abi mechanism may be a polypeptide or a polynucleotide such as a transcript (RNA), referred to as abortive infection (Abi) polypeptide/protein and abortive infection (Abi) polynucleotide such as abortive infection (Abi) transcript. The characteristics Abi mechanisms are shown in FIG. 1.

As indicated above the modes of action of the different Abi systems is very diverse, which is most likely the reason for the very low degree of protein homology that exists between the different Abi mechanisms.

The inventors provide a transconjugated lactic acid bacterium, wherein the transconjugated genetic material is chromosomal DNA encoding a genetic determinant for a phage resistance mechanism (φrm).

Further, the present inventors provide polynucleotides and abortive infection AbiV polypeptides or polynucleotides and bacteria cells expressing the same for use as donors to generate transconjugated bacteria with the abiV phenotype, wherein said phenotype is obtained by transconjugation of chromosomal DNA encoding and expression said AbiV.

The genetic determinant(s) for the Abi mechanism encodes at least one gene product, which upon expression in the a lactic acid bacterium such as a Lactococcus confers the abortive infection mechanisms, the Abi phenotype. The Abi mechanism may involve multiple components encoded by the host cell, which may work in synergy with polypeptides or nucleic acids encoded by the phage. However, a determinant of the Abi defence system is encoded by at least (and most often) one gene located in the Abi-loci. Accordingly, the genetic determinant of the AbiV system is encoded by the AbiV gene of the AbiV locus. The gene product encoded by orf1 (SEQ ID NO: 1) of the AbiV gene locus is the abortive infective infection protein AbiV. Expression of abortive infection protein AbiV in the host bacteria such as the transconjugants of the invention confers the AbiV mechanism identified by the AbiV phenotype.

Accordingly, in one embodiment of the present invention, the lactic acid bacteria comprise a chromosomal encoded phage resistance mechanism (such as an Abi mechanism), wherein said phage resistance mechanism is conferred by expression of a genetic determinant(s) for the said phage resistance mechanism, for example expression of AbiV gene to confers the AbiV phenotype

In second embodiment, the phage resistance mechanism is an Abi mechanism selected from the group consisting of AbiA, AbiF, AbiK, AbiP, AbiR, AbiT, AbiC, AbiE, Abil, AbiQ, AbiB, AbiD1, AbiU, AbiZ and AbiV and the determinant is encoded an expressed from at least one gene in the Abi loci (such as the AbiV).

In one embodiment, said transconjugant is a Lactococcus transconjugant.

In a second embodiment, the phage resistance mechanism transconjugated genetic material is a chromosomal DNA encoding at least one determinant of said phage resistance mechanism. The conjugated chromosomal DNA encoding said at least one determinant of said phage resistance mechanism is obtained from donor bacteria.

In a further embodiment, the phage resistance is conferred to a lactic acid bacterium by expression of the genetic determinant of said the phage resistance mechanism (such as the Abi gene), the product of said gene expression selected from a polypeptide or a polynucleotide such as a RNA transcript. In case, the genetic determinant(s) of said the phage resistance mechanism is silent the gene(s) may be activated e.g. insertional activation by a transposon such as Ghost9::ISS1 or spontaneously activated by selection for phage resistant mutants and subsequent screening with appropriate phage species.

In one embodiment of the invention, conjugated chromosomal DNA encoding the genetic determinant(s) of said the phage resistance mechanism is transcriptional active (and protein expressed) in the donor cell prior to transconjugation.

Further, expression of the genetic determinant(s) of said the phage resistance mechanism is sustained in the transconjugant receiving said chromosomal DNA encoding the genetic determinant(s). Thus, a bacterium encoding said the genetic determinant(s) of said the phage resistance mechanism may be identified and selected for expression of the genetic determinant(s) and subsequently used as donors for transconjugation of said activated genetic determinant(s) to a recipient bacterium (such as a lactic acid bacterium, such as a Lactococcus) to obtain a transconjugant of said recipient bacteria, wherein said transconjugant acquires the phage resistance phenotype of the donor cells.

### Lactococcus

Lactococcus is a lactic acid bacterial genus of five major species formerly included as members of the genus Streptococcus Group N and related species. They are gram-positive bacteria, and they are typically spherical or ovoid, 0.5-1.2 μm by 0.5-1.5 μm, and occur in pairs and short chains. They are non-spore forming and are not motile. The type species for the genus is L. lactis which in addition have two subspecies lactis and cremoris. Lactococcus is commonly used in the dairy industry in the manufacture of fermented dairy products. They can be used in single strain starter cultures, or in mixed strain cultures comprising other strains of Lactococcus or lactic acid bacteria such as e.g. Leuconostoc, Lactobacillus and Streptococcus.

Thus, in one embodiment of the present invention the lactic acid bacterium is selected from the group consisting of as Lactococcus, Streptococcus, Lactobacillus, Leuconostoc, Pediococcus. In a further embodiment, the lactic acid bacterium is a L. lactis such as the subspecies lactis or cremoris.

### A Fragment

A fragment according to the present invention is herein defined as a fragment of a polypeptide being at least 100 amino acids, preferably at least 110, more preferably at least 120 amino acids. With regards to SEQ ID NO 1, the fragment is preferably at least 100 amino acids in length, more preferably at least 125 amino acids in length, more preferably at least 150 amino acids in length, more preferably at least 175 and most preferably at least 190 amino acids in length.

### Promoter

The term “promoter”, “promoter region” or “promoter sequence” refer generally to transcriptional regulatory regions of a gene, which may be found at the 5′ or 3′ side of the coding region, or within the coding region, or within introns. As used herein the term promoter shall include any portion of genomic DNA (including genomic DNA disclosed herein), which is capable of initiating expression of operably linked nucleotide sequences at levels detectable above background. In the context with the present invention a “strong promoter” shall be understood as a promoter which results in expression of a polypeptide according to the invention, wherein the level of expression is significantly higher compared to the endogenous homologous promoter in the Lactococcus genome. The level of expression can be detected and/or measured by e.g. Northern blot, real-time PCR, reporter gene assays, etc. Thus, activation of the genetic determinant of the φrm of the invention refers to transcriptional activation of said genetic determinant leading to expression of a gene product conferring the φrm associated phage resistant phenotype to the bacterium.

In one embodiment, the φrm is activated in a donor bacterium before transconjugating the φrm to a lactic acid bacterium (recipient). Such donor may be obtained using a method for isolating bacterial mutants which have increased expression of a previously identified φrm (eg. an Abi) located on the genome preferably without using genetic modification.

### Expression Vector

A vector is a component or composition for facilitating cell transduction or transfection by a selected nucleic acid, or expression of the nucleic acid in the cell. Vectors include, e.g., plasmids, cosmids, viruses, BACs, PACs, P1, YACs, bacteria, poly-lysine, as well as linear nucleotide fragments etc. An “expression vector” is a nucleic acid construct or sequence, generated recombinantly or synthetically, with a series of specific nucleic acid elements that permit transcription of a particular nucleic acid sequence in a host cell. The expression vector can be part of a plasmid, virus, or nucleic acid fragment. The expression vector typically includes a nucleic acid to be transcribed operably linked to a promoter. The nucleic acid to be transcribed is typically under the direction or control of the promoter. The expression vector may replicate autonomously in the host cell or may integrate into the host genome after the transfection or transduction and replicate as part of the genome. Finally “an expression vector” encoding more than one polypeptide sequences according to the present invention comprises the situation wherein one expression vector comprises polynucleotide sequences encoding more than one polypeptide product as well as the situation wherein the polynucleotide sequences are cloned into two different expression vectors.

pGhost9::ISS1

The term “pGhost9::ISS1” covers a vector with an antibiotic resistance marker, a 30 Lactococcus replicon, and preferably also an E. coli replicon. The replicon is thermosensitive allowing for selection for integration into the host chromosome. Also the vector contains an insertion sequence that enables random integration of the vector into the host chromosome. It follows that vectors with similar functions may be used in connection with the present invention.

### Identity

The term “identity” or “sequence identity” is a measure of the degree of identity between polynucleotide sequences on a nucleotide-by-nucleotide basis or amino acid-by-amino acid basis, respectively over a window of comparison. Thus, the term “sequence identity” indicates a quantitative measure of the degree of homology between two amino acid sequences or between two nucleic acid sequences of equal length. If the two sequences to be compared are not of equal length, they must be aligned to give the best possible fit, allowing the insertion of gaps or, alternatively, truncation at the ends of the polypeptide sequences or nucleotide sequences. The sequence identity can be calculated as

$$\frac{( {N}_{ref} - {N}_{dif} ) 100}{{N}_{ref}} ,$$

wherein Ndif is the total number of non-identical residues in the two sequences when aligned and wherein Nref is the number of residues in one of the sequences. Hence, the DNA sequence AGTCAGTC will have a sequence identity of 75% with the sequence AATCAATC (Ndif=2 and Nref=8). A gap is counted as non-identity of the specific residue(s), i.e. the DNA sequence AGTGTC will have a sequence identity of 75% with the DNA sequence AGTCAGTC (Ndif=2 and Nref=8). With respect all embodiments of the invention relating to nucleotide sequences, the percentage of sequence identity between one or more sequences may also be based on alignments using suitable software or online applications such as the clustalW software (http:/www.ebi.ac.uk/clustalW/index.html) with default settings. For nucleotide sequence alignments these settings are: Alignment=3Dfull, Gap Open 10.00, Gap Ext. 0.20, Gap separation Dist. 4, DNA weight matrix: identity (IUB).

Alternatively, and as illustrated in the examples, nucleotide sequences may be analysed using programme DNASIS Max and the comparison of the sequences may be done at using suitable software or online applications such as http://www.paralign.org/. This service is based on the two comparison algorithms called Smith-Waterman (SW) and ParAlign. The first algorithm was published by Smith and Waterman (1981) and is a well established method that finds the optimal local alignment of two sequences. The other algorithm, ParAlign, is a heuristic method for sequence alignment; details on the method is published in Rognes (2001). Default settings for score matrix and Gap penalties as well as E-values were used.

Sequences according to the present invention have an identity of at least 70% to SEQ ID NO 1, or a fragment thereof.

### Starter Culture

The term “starter culture” refers to a culture of bacteria for the use of initiating a fermentation of raw material or semi-manufacture. The starter cultures are typically used for the starting the fermentation of raw material or semi-manufacture such as milk in the preparation of a food product such as a dairy product. The starter culture may comprise a composition of bacterial species suitable for the fermentation process in question such as lactic acid bacteria. Alternatively, the starter culture is based on a single bacterial species.

### Food Products

Food products according to the present invention include milk based products that have been subject to fermentation processes. Examples thereof include: sour cream, crème fraîche, buttermilk, butter, cheese, cottage cheese, quark, cream cheese, fromage frais, yoghurt, etc. However, other types of food products may also be produced using fermentation or fermentative microorgansims according to the present invention such as e.g fruit juices, fermented vegetables/fruits, processed meat products, etc.

### Transconjugation, Transconjugant

The term transconjugation refers to the transfer of material from one bacterium or another (conjugal transfer). The transconjugation is not regarded as a process of genetic engineering. Accordingly, a transconjugant bacterium is not regarded as a genetic modified organism (GMO) unless otherwise modified by means of genetic engineering. The transconjugation may involve transfer of plasmids from the donor to the recipient bacterium. The transconjugation involves transfer of chromosomal DNA. The recipient of the genetic material is subsequently referred to as a transconjugant. A chromosomal transconjugant refers to a bacterium, which has received genetic material in the form of chromosomal DNA from a donor bacterium.

Only a few abi mechanisms have been isolated from the chromosome of L. lactis. This may partly be due to the fact that it is generally easier to isolate genes present on plasmids compared to isolation of genes present on chromosomes. Due to a high mutation rate in the phage populations leading to mutant phages which are insensitive to applied φrms, there is a constant need for the dairy industry to find new ways to protect their production strains. Consumer skepticism and legislative issues make the use of genetically modified organisms (GMO) undesirable. Therefore isolation of novel natural φrms and subsequent activation and natural transfer to production strains are industrially important. The procedure used in the present invention to activate a φrm from the chromosome of a lactic acid bacteria such as L. lactis and subsequently transfer the φrm can generally be used to activate and transfer φrms on bacterial chromosomes.

By isolating spontaneous phage resistant mutants with a similar phenotype with regards to efficiency against a range of appropriate phage species it is possible to identify strains expressing a given previously identified φrm without having to use genetic modification. Using this method, non-GMO phage resistant strains can thus be isolated. Use of non-GMO starter cultures may be an advantage in some case, in particular in relation to the fact that the legislation in some countries does not allow use of GMO. Furthermore, some consumers tend to prefer non-GMO derived products.

The present invention thus relates to an isolated polynucleotide sequence that encodes a polypeptide with at least 70% identity, preferably at least 75% identity, more preferably at least 80% identity, more preferably at least 85% identity, more preferably at least 70% identity, preferably at least 75% identity, more preferably at least 80% identity, more preferably at least 85% identity, more preferably at least 90% identity, and most preferably at least 95% identity with SEQ ID NO 1 (AbiV from Lactococcus lactis), or a fragment thereof, and wherein expression of said polynucleotide confers at least one phage resistance mechanism to a Lactococcus bacterium. This polynucleotide sequence is found naturally in the Lactococcus bacterium, but it is normally not transcriptionally active. It has surprisingly been found that expression of this polypeptide confers a previously unknown phage resistance mechanism to the bacterium.

The present invention also relates to an isolated polynucleotide derived from a Lactococcus lactis phage that encodes a polypeptide with at least 70% identity, preferably at least 75% identity, more preferably at least 80% identity, more preferably at least 85% identity, more preferably at least 95% identity, preferably at least 97% identity, and most preferably at least 99% identity with SEQ ID NO 2, or a fragment thereof and/or an isolated polynucleotide that encodes a polypeptide with at least 70% identity, preferably at least 75% identity, more preferably at least 80% identity, more preferably at least 85% identity, more preferably at least 95% identity, preferably at least 97% identity, and most preferably at least 99% identity with SEQ ID NO 7. Optionally, the polynucleotide sequence may encode at least one of SEQ ID NO 1, SEQ ID NO 2, and SEQ ID NO 7 or any variant thereof in the form of one or more polynucleotide sequences.

SEQ ID NO 2 and SEQ ID NO 7 are phage proteins. The inventors have found out that these proteins most likely need to be mutated in order for the phage to escape the phage resistance mechanism conferred by expression of SEQ ID NO 1 or variants thereof. Phage proteins of the infecting phage according to the present invention therefore have at least 70% identity with SEQ ID NO 2 and/or SEQ ID NO 7 in order to provide functional phage protein that may suppress the effects of emergence of mutated phage protein that could potentially suppress the effects of the translated SEQ ID NO 1 protein or variants thereof. In a preferred embodiment according to the present invention, polynucleotide sequences encoding both SEQ ID NO 1 or variants thereof as well as SEQ ID NO 2 and/or SEQ ID NO 7 or variants thereof, are thus provided thus conferring highly efficient phage protection mechanisms to a host cell. It furthermore follows that the invention relates to expression vectors as well as Lactococcus bacteria and/or starter cultures comprising polynucleotide sequences encoding such polypeptide sequences.

The present invention further relates to an isolated polypeptide conferring at least one phage resistance mechanism to a Lactococcus bacterium, wherein said polypeptide is selected from one or more of the group consisting of: a polypeptide with at least 70% identity with SEQ ID NO 1, or a fragment thereof, a polypeptide with at least 70% identity with SEQ ID NO 2, or a fragment thereof, and a polypeptide with at least 70% identity with SEQ ID NO 7, or a fragment thereof.

The present invention also relates to the use of one or more polynucleotides according to the present invention and/or one or more polypeptides according to the present invention for improving phage resistance in a Lactococcus bacterium.

The present invention also relates to a method for fermenting food product, said method comprising the step of adding one or more of the components according to the present invention. The invention furthermore relates to products that can be obtained and/or are obtained using this method.

The present invention also relates to a method for obtaining phage resistant bacterial cells, said method comprising use of pGhost9::ISS1 (or similar systems) for random insertion into a bacterial cell and subsequently screening and selecting for phage resistant cells. The invention furthermore relates to cells that can be obtained and/or are obtained by such methods. In a preferred embodiment, the cell is a Lactocuccus bacterium wherein a polynucleotide encoding SEQ ID NO 1 (or a variant thereof) is transcriptionally active. The phage resistant bacteria obtained by this method may be used as donor for transconjugation chromosomal DNA encoding the activated the genetic determinant(s)

The present invention also relates to a transconjugant lactic acid bacterium such as a Lactococcus bacterium that expresses at least one polypeptide selected from the group consisting of: a polypeptide with at least 70% identity with SEQ ID NO 1, or a fragment thereof, a polypeptide with at least 70% identity with SEQ ID NO 2, or a fragment thereof, and a polypeptide with at least 70% identity with SEQ ID NO 7, or a fragment thereof.

The present invention also relates to a lactic acid bacterium such as Lactococcus bacterium in which a silent (non-expressed) φrm has been identified and in which isolation of sponataneous phage resistant mutants are screened and compared to the phage typing pattern of the isolated φrm. Spontaneous bacterial mutants with the same phage typing pattern are likely to naturally have activated the desired φrm thereby obtaining a non-GMO mutant with the desired φrm phenotype.

### Transconjugants of a Lactic Acid Bacterium Such as Lactococcus Bacterium

A first aspect of the present invention relates to a transconjugated lactic acid bacterium, wherein the transconjugated genetic material is chromosomal DNA encoding a genetic determinant for a phage resistance mechanism (φrm).

In one embodiment, said genetic determinant for a phage resistance mechanism (φrm) is expressed. In a further embodiment, said genetic determinant was activated in a donor cells prior transconjugating the chromosomal DNA encoding a genetic determinant for a phage resistance mechanism (φrm) to the recipient cell.

In a second embodiment, the lactic acid bacteria is a Lactococcus transconjugant such as Lactococcus is Lactococcus Lactis.

In a third embodiment, the phage resistance mechanism is an abortive infection mechanism (Abi). In a further embodiment, said abortive infection mechanism (Abi) is selected from the group consisting of AbiA, AbiF, AbiK, AbiP, AbiR, AbiT, AbiC, AbiE, Abil, AbiQ, AbiB, AbiD1, AbiU, AbiZ and AbiV. In a preferred embodiment, the said abortive infection mechanism (Abi) is AbiV.

In another embodiment, said genetic determinant for said phage resistance mechanism is encoded by a polynucleotide sequence that encodes a polypeptide with at least 70% identity with SEQ ID NO 1, or a fragment thereof, and wherein expression of said polynucleotide confers at least one phage resistance mechanism to a transconjugated lactic acid bacterium.

A second aspect of the present invention relates to a starter culture composition comprising a transconjugated lactic acid bacterium of the invention. The starter culture may be used in a method for preparing a fermented food product.

Accordingly, one aspect relates to a method for preparing a fermented food product, said method comprising adding to the raw material or semi-manufacture to be fermented at least one of the components selected from the list consisting of: a transconjugated lactic acid bacterium according any of the invention, a starter culture according to the invention.

A further aspect relates to a food product comprising a transconjugated lactic acid bacterium according to invention. In one embodiment, said transconjugated lactic acid bacterium is a Lactococcus transconjugant such as Lactococcus is Lactococcus Lactis. In a further embodiment, food product is a dairy product Yet a further aspect relates to the use of a transconjugated lactic acid bacterium according the any of the preceding claims in the production of a food product. In one embodiment, said product is a diary product.

A further embodiment relates to a method of preparing a transconjugated lactic acid bacterium according to the invention, said method comprising culturing a donor bacterium comprising chromosomal DNA encoded said genetic determinant for said phage resistance mechanism and a recipient lactic acid bacterium in a medium suitable for transconjugation, and subsequently isolating a transconjugated lactic acid bacterium conferring said phage resistance. In one embodiment, said donor comprises a chromosomal encoded phage resistance mechanism according to invention. In one embodiment, said donor comprises a chromosomal genetic determinant for a phage resistance mechanism according to the invention. In a particular embodiment, said phage resistance is AbiV.

In a further embodiment, said genetic determinant for phage resistance is coupled to a selection marker used for selection of a transconjugated lactic acid bacterium. In one embodiment, said selection marker is the erythromycin resistance gene (Erm^{r}).

One aspect of the invention relates to the use of a bacterium comprising a chromosomal genetic determinant for a phage resistance mechanism (φrm) as donor for the preparation of a transconjugant bacterium comprising a chromosomal encoded phage resistance mechanism. In one embodiment, said genetic determinant for a phage resistance mechanism (φrm) is expressed.

In a further embodiment, wherein said bacterium was obtained by selection for spontaneous activation of said genetic determinant for a phage resistance mechanism (φrm). In yet an embodiment, said genetic determinant for a phage resistance mechanism (φrm) is mechanism is an abortive infection mechanism (Abi), such as an abortive infection mechanism (Abi) is selected from the group consisting of AbiA, AbiF, AbiK, AbiP, AbiR, AbiT, AbiC, AbiE, Abil, AbiQ, AbiB, AbiD1, AbiU, AbiZ and AbiV.

A further aspect of the present invention relates to a transconjugated lactic acid bacterium obtained by a method according the invention. In one embodiment, said lactic acid bacteria is a Lactococcus such as Lactococcus Lactis.

One aspect of the invention relates to transconjugants of a lactic acid bacteria such as a Lactococcus comprising a chromosomal encoded phage resistance mechanism. In one particular embodiment the Lactococcus is Lactococcus lactis.

In a preferred embodiment the transconjugant of a lactic acid bacteria such as a Lactococcus transconjugant is obtained by transconjugation of chromosomal DNA encoding the phage resistance mechanism of the invention. Accordingly, the chromosomal DNA encoding the phage resistance mechanism of the invention originates from a donor bacterium and is transferred to a recipient bacterium such as a Lactococcus species by transconjugation. The transconjugant of a lactic acid bacteria such as a Lactococcus transconjugant thus comprises a chromosomal encoded phage resistance mechanism and confers resistance to the infection of a least one species of phages. The donor bacterium of the invention were sensitive to infection by a least one species of phages prior to the transconjugation. In one embodiment the lactic acid bacteria such as a Lactococcus is semi-resistent to a least one species of phages.

In one embodiment, the transconjugant of a lactic acid bacteria such as a Lactococcus transconjugant, wherein the transconjugated genetic material is a chromosomal encoded phage resistance mechanism.

In one particular embodiment, the transconjugant of a lactic acid bacteria such as a Lactococcus transconjugant the chromosomal encoded phage resistance mechanism is an abortive infection mechanism (Abi). Accordingly, the transconjugated genetic material comprises nucleic acids sequences encoding a phage resistance mechanism. According to the invention a phage resistance mechanism may involve other components (nucleic acids or polypeptides) than those encoded by the transconjugated genetic material.

For example, the present invention relates to use of SEQ ID NO 1 for conferring phage resistance to bacterial cells. A transconjugant of a lactic acid bacteria such as a Lactococcus transconjugant comprising SEQ ID NO 1 in combination with SEQ ID NO 2 is resistant to phage infection. Thus, in one embodiment, the nucleic acid or polypeptide encoded by the transconjugated genetic material may activate a phage resistance mechanism independent of components encoded by the genetic material of the donor bacterium or infecting phage. In another embodiment, the nucleic acid or polypeptide encoded by the transconjugated genetic material may activate a encoding a phage resistance mechanism working in combination with of components encoded by the genetic material of the donor bacterium or infecting phage.

In one embodiment, the chromosomal encoded phage resistance mechanism is a phage resistance mechanism selected from the group consisting of a phage adsorption interference mechanism, a phage DNA injection interference system, a restriction/modification system (R/M) and an abortive infection mechanisms (Abi).

In a further embodiment of the invention, the chromosomal encoded phage resistance mechanism is an abortive infection mechanism (Abi). Accordingly, in another embodiment of the invention the abortive infection mechanism (Abi) is selected from the group consisting of AbiA, AbiF, AbiK, AbiP, AbiR, AbiT, AbiC, AbiE, Abil, AbiQ, AbiB, AbiD1, AbiU, AbiZ and AbiV. In a yet another embodiment the abortive infection mechanism (Abi) is AbiV.

In a preferred embodiment, the chromosomal encoded phage resistance mechanism is encoded by a polynucleotide sequence that encodes a polypeptide with at least 70% identity with SEQ ID NO 1, or a fragment thereof, and wherein expression of said polynucleotide confers at least one phage resistance mechanism to a lactic acid bacterium such as a Lactococcus bacterium.

The transconjugants of the invention may be used in the preparation of starter cultures. In one aspect of the invention, a starter culture composition comprises a transconjugant of the invention such as a transconjugant Lactococcus. In a particular embodiments, the starter culture comprises a Lactococcus transconjugant encoded an abortive infection mechanism (Abi) such as AbiV.

The transconjugant of the invention such as a Lactococcus transconjugant of the invention may be used in the process involving fermentation such fermentation of raw material or semi-manufacture in the preparation of a food product. One aspect of the invention relates to a method for preparing a fermented food product, said method comprising adding to the raw material or semi-manufacture to be fermented at least one of the components selected from the list consisting of: a transconjugant of the invention (such as a Lactococcus transconjugant), or a starter culture of the invention.

An aspect of the invention relates to the use of a transconjugant of the invention in the production of a food product, for example a dairy product. In one embodiment, the transconjugant is a Lactococcus transconjugant.

Accordingly, another aspect of the invention relates to a food product comprising a Lactococcus transconjugant of the invention. In one embodiment, the food product is a dairy product.

The present invention further provides methods of preparing a transconjugant bacterium such as a transconjugant Lactococcus species, which upon transconjugation confers resistance to infection at least species of phage.

One aspect of the invention relates to a method of preparing a Lactococcus transconjugant comprising a chromosomal encoded phage resistance mechanism comprising culturing a donor bacterium comprising a chromosomal encoded phage resistance mechanism and a recipient Lactococcus bacterium in a medium suitable for transconjugation, and subsequently isolating a transconjugated Lactococcus transconjugant conferring phage resistance. In another embodiment, the donor comprises a chromosomal encoded phage resistance mechanism of the present invention, for example abiV. An example of such method are disclosed in Example 21, which also disclose transconjugants obtained by such methods.

In a particular embodiment the chromosomal encoded phage resistance is coupled to a selection marker used selection of Lactococcus transconjugant. The proximity of a chromosomal selection marker to the chromosomal encoded phage resistance allows for the selection of transconjugation events involving the co-transfer of the chromosomal encoded phage resistance and a chromosomal selection marker. In one embodiment, the selection marker is the erythromycin resistance gene (Erm^{r}).

Another aspect of the present invention relates to the use of bacterium comprising a chromosomal encoded phage resistance mechanism as donor for the preparation of a transconjugant bacterium comprising a chromosomal encoded phage resistance mechanism such as a donor comprises a chromosomal encoded phage resistance mechanism of the invention. Accordingly, final aspect of the invention concerns a Lactococcus transconjugant comprising a chromosomal encoded phage resistance mechanism obtained by a method of the invention.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

### Bacterial Strains, Plasmids, and Media

Strains and plasmids used in this invention are listed in table 1. Escherichia coli was grown at 37° C. in LB medium. Lactococcus lactis was grown in M17 with the supplement of 0.5% glucose (GM17). Lactococci were grown at 30° C. except strains containing the thermo sensitive vector pGhost9::ISS1. These strains were grown at 28° C. for replication of the vector or 36° C. to avoid replication. When appropriate, antibiotics were added as follows: E. coli, 100 μg/ml of ampicillin, 10 μg/ml of chloramphenicol, 150 μg/ml of erythromycin; for L. lactis, 5 μg/ml of chloramphenicol, 3 μg/ml of erythromycin.

TABLE 1
List of bacteria, phages and plasmids used in the invention

| Bacterial strain, | | |
|---|---|---|
| phage or plasmid | Characteristic | Source |
| Lactococcus | | |
| lactis | | |
| SMQ-86 | Lactococcus lactis subsp. cremoris. Multiple plasmids, pSA3, host for the tested P335 phages. Erm^{R} | (2) |
| IL1403 | Lactococcus lactis subsp. lactis IL1403, host for some 936 phages | (1) |
| MB112 | Lactococcus lactis subsp. cremoris MG1363, Δupp, Host for 936 and c2 phages | (7) |
| JH-20 | MB112 (pJH2); Cam^{R}, Abi⁺ | This Invention |
| JH-22 | IL1403 (pJH2); Cam^{R}, Abi⁺ | This Invention |
| JH-23 | SMQ-86 (pJH2); Cam^{R}, Abi⁺ | This Invention |
| JH-24 | MB112 (pJH3); Cam^{R}, Abi⁻ | This Invention |
| JH-25 | MB112 (pJH4); Cam^{R}, Abi⁺ | This Invention |
| JH-26 | MB112 (pJH5); Cam^{R}, Abi⁺ | This Invention |
| JH-32 | MB112 (pGhost9::ISS1 inserted in Lin at bp1962); Erm^{R}, grown at 36° C., Abi⁺ | This Invention |
| JH-46 | MB112 (pGhost9::ISS1 inserted in Lin at bp1962); Erm^{R}, grown at 36° C., Abi⁺ | This Invention |
| JH-47 | MB112 (pGhost9::ISS1 inserted in Lin at bp2296); Erm^{R}, grown at 36° C., Abi⁺ | This Invention |
| JH-48 | MB112 (pGhost9::ISS1 inserted in Lin at bp2240); Erm^{R}, grown at 36° C., Abi⁺ | This Invention |
| JH-49 | JH-32 cured for pGhost9, leaving ISS1 in place; Abi⁻ | This Invention |
| JH-50 | JH-46 cured for pGhost9, leaving ISS1 in place; Abi⁻ | This Invention |
| JH-51 | JH-47 cured for pGhost9, leaving ISS1 in place; Abi⁻ | This Invention |
| JH-52 | JH-48 cured for pGhost9, leaving ISS1 in place; Abi⁻ | This Invention |
| JH-53 | MB112 (pJH6); Cam^{R}, Abi⁻ | This Invention |
| JH-54 | MB112 (pLC5); Cam^{R}, Abi⁻ | This Invention |
| JH-80 | MB112 (spontaneous mutation to express φrm); Abi⁺ | This Invention |
| JH-81 | transconjugant with transferred φrm and Erm^{R} from JH-32 to LKH208; Abi⁺, Erm^{R}, Rif^{R}, Strep^{R}, FU^{S} | This Invention |
| MG1614 | MG 1363 Rif^{R}, Strep^{R} (M. Gasson) | (5) |
| Escherichia coli | | |
| EC1000 | RepA⁺ MC1000, Km^{R} | (4) |
| JH-56 | EC1000 (pJH7), Erm^{R} | This Invention |
| JH-57 | EC1000 (pJH8), Erm^{R} | This Invention |
| JH-58 | EC1000 (pJH9), Erm^{R} | This Invention |
| JH-59 | EC1000 (pJH10), Erm^{R} | This Invention |
| TOP10F′ | Chemically competent cells from the TOPO TA cloning kit | Invitrogen |
| JH-19d | EC1000 (pJH6), Cam^{R} | This Invention |
| phages | | |
| p2 | Small isometric headed, 936 species | S.M |
| sk1 | Small isometric headed, 936 species | F.V |
| jj50 | Small isometric headed, 936 species | F.V |
| 712 | Small isometric headed, 936 species | S.M |
| P008 | Small isometric headed, 936 species | S.M |
| bIL170 | Small isometric headed, 936 species | S.M |
| c2 | Prolate headed, c2 species | S.M |
| bIL67 | Prolate headed, c2 species | S.M |
| ml3 | Prolate headed, c2 species | S.M |
| eb1 | Prolate headed, c2 species | S.M |
| ul36 | Small isometric headed, P335 species | S.M |
| KITI | Small isometric headed, P335 species | S.M |
| Ø31 | Small isometric headed, P335 species | S.M |
| Ø50 | Small isometric headed, P335 species | S.M |
| Q33 | Small isometric headed, P335 species | S.M |
| Q30 | Small isometric headed, P335 species | S.M |
| P335 | Small isometric headed, P335 species | S.M |
| p2.1 | Small isometric headed, 936 species, deletion in orf26 | This Invention |
| sk1.1 | Small isometric headed, 936 species, nonsense mutation in orf26 | This Invention |
| jj50.1 | Small isometric headed, 936 species, nonsense mutation in orf25 (homologue to p2 orf26) | This Invention |
| P008.1 | Small isometric headed, 936 species, nonsense mutation in orf33 (homologue to p2 orf26) | This Invention |
| bIL170.1 | Small isometric headed, 936 species, nonsense mutation in e24 (homologue to p2 orf26) | This Invention |
| c2.1 | Prolate headed, c2 species, mutation in e11 (homologue to p2 orf26) | This Invention |
| plasmids | | |
| pCI372 | Shuttle vector for E. coli and L. lactis. No promoter in front of multiple cloning site; Cam^{R} | (3) |
| pLC5 | Expression vector for L. lactis and E. coli. Promoter in front of PstI site used for cloning; Cam^{R} | This Invention |
| pGhost9::ISS1 | pGhost9::ISS1, temperature sensitive vector with insertion sequence used for random mutagenesis, | (6) |
| | Erm^{R} | |
| pJH2 | bp 1021-2320 on Lin sequence* cloned in PstI site of pLC5; Cam^{R} | This Invention |
| pJH3 | pJH2, restricted in ClaI site of AbiV and filled with Klenow, gives frameshift mutation; Cam^{R} | This Invention |
| pJH4 | pJH2 isolated from JH22 | This Invention |
| pJH5 | pJH2 isolated from JH23 | This Invention |
| pJH6 | bp 1021-2320 on Lin sequence* cloned in PstI and XbaI sites of pCI372 | This Invention |
| pJH7 | HindIII rescue of pGhost9::ISS1 with flanking chromosomal DNA from JH-32 | This Invention |
| pJH8 | HindIII rescue of pGhost9::ISS1 with flanking chromosomal DNA from JH-46 | This Invention |
| pJH9 | HindIII rescue of pGhost9::ISS1 with flanking chromosomal DNA from JH-47 | This Invention |

Lin sequence refers to GeneBank acc nr AF324839
Cam^{R}, chloramphenicol resistance;
Amp^{R}, ampicillin resistance;
Erm^{R}, Erythromycin resistance;
Km^{R}, Kanamycin resistance;
Rif^{R}, Rifampicin resistance;
Strep^{R}, Streptomycin resistance;
FU^{R} fluorouracil resistance
Abi⁺, phage resistance phenotype;
Abi⁻, phage sensitive phenotype
F.V = Finn K. Vogensen, University of Copenhagen
S.M = Sylvain Moineau

### Example 2

### Bacteriophage Propagation and Assays

Bacteriophages used in this invention are listed in table 1. Bacteriophages sk1 and jj50 were kindly provided by F. K. Vogensen (University of Copenhagen). Prior to use all phages were purified two times by picking a single plaque with a sterile Pasteur pipette and plating it on a sensitive host. Propagation of phages to obtain high titer lysates was performed in two steps:

In the first propagation a single plaque was transferred into a fresh ON culture of a sensitive host inoculated (1%) in GM17 supplemented with 10 mM CaCl₂ and incubated at 30° C. (or 36° C. in the case of pGhost9::ISS1 containing host strains) until lysis. The lysate was filtered through a 0.45 μm syringe filter.

The second propagation was performed by inoculating an exponentially growing host culture at OD₆₀₀=0.2 with phages from the first propagation (10⁴ pfu/ml) in GM17+10 mM CaCl₂.

The culture was then incubated with agitation (200 rpm) until lysis at the same temperature as for the first propagation. The lysate was filtered (0.45 μm filter). The titer of phage lysates was determined using conventional methods.

Efficiency of plaquing (EOP) was calculated by dividing the titer on the tested strain with the titer on the sensitive wt strain. Adsorption assays were conducted as described by Sanders and Klaenhammer (17) except a 5 min incubation period was used instead of 15 min. Cell survival was assayed by the method of Behnke and Malke (2) using a multiplicity of infection (MOI) of 5. One-step growth assay (and determination of burst size) and center of infection (COI) assay was performed as described previously (14) by using MOIs of 0.2 and 0.5, respectively. ECOI (efficiency of COI) was determined by dividing the number of COI from the resistant strain by the number of COI from the sensitive strain. Replication of phage DNA was followed in a time course experiment using the method of Hill et al. (8). Visualization of phage DNA by labeling with the fluorescent dye SYBR-Gold was performed as described by Noble and Fuhrman (15) with the following modifications: The original SYBR-Gold solution was diluted (×1000). Phage lysate to be stained was treated with 1 μg/ml DNAse and RNAse and incubated for 30 min at 37° C. The lysate was stained with the diluted SYBR-Gold to give 2.5% final concentration (vol/vol) diluted SYBR-Gold and left ON at 4° C. in the dark. One μl of the labeled phage stock was mixed with 1 μl exponentially growing cell culture and visualized under a Zeiss axioplan epifluorescence microscope.

### Example 3

### Mutagenesis with pGhost9::ISS1

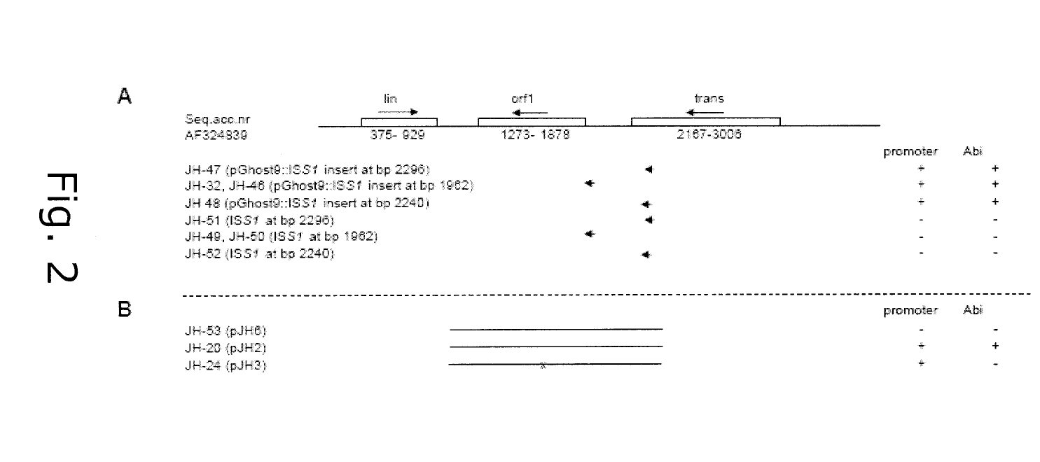

Random integration of the vector pGhost9::ISS1 into the chromosome of MB112 and subsequent cloning of flanking chromosomal DNA was performed essentially using the method of Maguin et al. (10). The method of Maguin, however, is normally used to identify inactivation of genes by randomly inserting the construct in chromosomal genes, thereby inactivating them. Subsequent selection for a desired phenotype enables screening for strains containing a loss of function mutation. The fact that all inspected mutants in the present invention had insertions in non coding regions or genes upstream of orf1 together with the observation that presence of the complete vector pGhost9::ISS1 was needed for the Abi⁺ phenotype led to the hypothesis that the abiV gene (orf1) was transcribed from the promoter encoding the erythromycin resistance gene in pGhost9::ISS1 (FIG. 2). Previous studies have reported promoter activity in the ISS1 sequence (5). No effect on phage resistance phenotype of such promoter activity was observed in the present invention. It has not previously been shown that random insertion of the vector and subsequent transcription from the promoter of the erythromycin resistance gene can be used to activate existing biological mechanisms, such as e.g. Abi-mechanisms.

To ensure that the mutations in the isolated strains had arisen from independent events, the integration step (growth at 37° C.) was performed on 12 separate cultures. After the integration step, the cultures were diluted ×10.000 in GM17+3 μg/ml Erythromycin and left for phenotypic expression ON at 37° C. These cultures were inoculated (1%) and when growing exponentially aliquots were removed. 10 mM CaCl₂ (final concentration) was added to these aliquots before inoculating with phage sk1 (MOI>1). After 10 min incubation at 37° C. the cultures were spread on selective GM17+Erm plates. A number of phage resistant colonies were isolated and purified from each of the 12 independent cultures. Four strains were chosen from independent cultures to identify the location of the inserted pGhost9::ISS1. This was performed by rescuing of the inserted vector and cloning of flanking chromosomal DNA. The cloned chromosomal DNA fragments were subsequently sequenced

### Example 4

### DNA Isolation and Manipulation

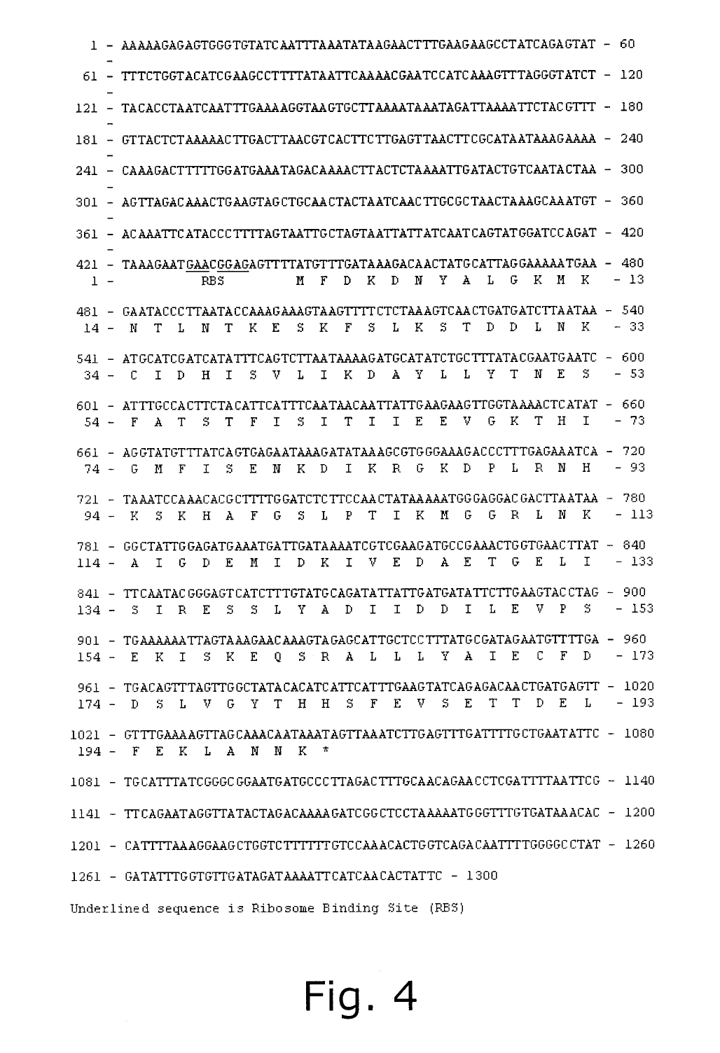

Plasmid DNA was isolated from E. coli and L. lactis using the QIAprep Spin Miniprep Kit (Qiagen); for L. lactis however, lysozyme (15 mg/ml) was added to buffer p1 and the solution with the resuspended cells was incubated at 37° C. for min before proceeding with the manufacturers protocol. Phage DNA was prepared using the Qiagen Lambda Maxi Kit (Qiagen) with the addition of proteinase K (20 mg/ml) to buffer L3 and subsequent incubation at 65° C. for 30 min before adding buffer L4. Total intracellular DNA was isolated using the method of Hill et al. (8). Restriction enzymes, T4 DNA ligase and Klenow fragment (Fermentas) were used according to the manufacturer's instructions. Electroporation of E. coli and L. lactis was performed as described previously (13). The DNA fragment corresponding to by 1021 to 2320 (FIG. 4) in the GenBank sequence AF324839 was subcloned in the TOPO TA cloning kit prior to cloning in pCI372 and pLC5.

### Example 5

### DNA Sequencing and Sequence Analysis of DNA and Protein

Oligonucleotide sequences used for plasmid constructions and sequencing: For sequencing the flanking chromosomal DNA of the rescued pGhost9::ISS1 inserts a primer located in the ISS1 was used (5′-GAAGAAATGGAACGCTC-3′).

Phage genome sequencing was performed with an ABI prism 3700 apparatus from the genomic platform at the research center of the Centre Hospitalier de l'Université Laval using a set of oligonucleotides previously used for sequencing of 936 phage genomes (11).

Sequence data was assembled using the Staden Pregap4 version 1.5. Sequence homology searches in databases were done using BLAST (1). Molecular weight and pI of the investigated proteins were estimated using the Protein Calculator at the website: http://www.scripps.edu/˜cdputnam/protcalc.html

### Example 6

### A Phage Resistance Mechanism (φrm) is Found on the Chromosome of Lactococcus lactis subsp. cremoris MG1363

L. lactis subsp. cremoris MG1363 is sensitive to infection of phages from the 936 and c2 species. In this invention a transposon mutagenesis system (described in details in (10)) was used to identify a novel φrm on the chromosome of MG1363. The system (pGhost::ISS1) comprises the vector pGhost9 containing an erythromycin resistance gene (Em^{r}) and the ISS1 insertion sequence which allows for random integration of the construct into the host chromosome. Due to a thermosensitive origin of replication (plasmid is not replicating at 37° C.) it is possible to select for mutants with the construct inserted in the chromosome by growing at 37° C. in the presence of erythromycin, allowing for phenotypic expression by growing at selective conditions ON.

This selection was done for a number of independently grown cultures resulting in isolation of independent integration events. These cultures were screened for resistance to phage sk1 by selecting colonies growing on erythromycin plates in the presence of phages (MOI>1). The frequency of mutations conferring phage resistance was 100 times higher in cultures with mutants containing the pGhost::ISS1 inserts compared to the control cultures in which the phage resistance was caused by spontaneous mutations. This clearly indicates that the mutations in the phage resistant mutants containing pGhost9::ISS1 in most cases were caused by the insertion of this construct.

A number of Em^{r}/φrm⁺ colonies were isolated. From four of these independently mutagenized cultures, the inserted construct was obtained along with a piece of flanking chromosomal DNA. Sequence analysis revealed insertions on the chromosome corresponding to by 1962 (strains JH-32 and JH-46), by 2240 (JH-48) and by 2296 (JH-47) on the sequence available in GenBank under the accession number AF324839 (hereafter designated Lin). Bp 1021 to bp2320 therein corresponds to SEQ ID NO 6. All strains had insertions in the intergenic region between two genes (designated orf1 and trans) or in the 3′ end of the trans gene (FIG. 2A). There are no genes in the same orientation immediately downstream of orf1 and since the mutagenizing constructs were all inserted in the same orientation pointing towards orf1 it was hypothesized that orf1 is encoding a φrm which is transcribed from the Em^{r} gene promoter when pGhost::ISS1 is inserted upstream of orf1. Curing the strains for the vector (leaving a single copy of ISS1 at the insertion site) resulted in φrm⁻ phenotype supporting the hypothesis that a promoter in pGhost::ISS1 is needed for transcription of orf1 and the resulting φrm⁺ phenotype. This implies that orf1 is silent in wt MG1363 which is supported by the phage sensitive phenotype of this strain.

### Example 7

### Identification of orf1 as a φrm

To test if orf1 is a φrm, a fragment corresponding to by 1021 to 2320 on the Lin sequence was cloned in the shuttle vector pCI372 (pJH6) and in the expression vector pLC5 (pJH2). These constructs were transformed in MB112 and the resulting strains (JH-53 and JH-20, respectively) were tested by cross streaking assay for resistance to phage p2. JH-53 containing pJH6 with no promoter upstream of orf1 showed no phage resistance phenotype. In comparison, JH-20 containing pJH2 with orf1 cloned downstream of a strong promoter revealed phage resistance phenotype.

To verify orf1 as being the φrm, a frameshift mutation was introduced in orf1 by filling a unique ClaI site with Klenow fragment followed by ligation and transformation of this vector (pJH3) in wt MB112. The mutated orf1 was sequenced verifying the frameshift mutation. The resulting strain JH-24 had a phage sensitive phenotype and it was therefore concluded that orf1 is encoding a φrm.

### Example 8

### The Isolated φrm is Effective Against Phages of the 936 and C2 Species

The three phage species 936, c2 and P335, known to be responsible for the majority of phage caused fermentation failures were tested for their sensitivity to the φrm. Four strains of the 936 species were tested against JH-20. Efficiency of plaquing (EOP) values around 10⁻⁴ were obtained for phages p2, sk1 and jj50 while phage 712 was insensitive to the φrm (Table 2). pJH2 was inserted into the host JH-22 (L. lactis subsp. lactis IL1403) which is sensitive to the 936 phages P008 and bIL170. When tested against these phages the φrm revealed EOP values around 10⁻⁴. Similar values were obtained when testing JH-20 against four phages of the c2 species (Table 2). Similar EOP values were obtained for MB112 and JH-54 when tested against the 936 and c2 phage species (data not shown), thus ruling out the possibility for the vector pLCS being responsible for the φrm⁺ phenotype.

TABLE 2

| | Phage | Host strain | EOP |
|---|---|---|---|
| 936 species^{a} | sk1 | JH-20 | 2.7 ± 1.4 × 10⁻⁴ |
| | p2 | JH-20 | 4.8 ± 1.8 × 10⁻⁴ |
| | jj50 | JH-20 | 8.3 ± 0.5 × 10⁻⁵ |
| | 712 | JH-20 | 1.1 ± 0.2 |
| | P008 | JH-22 | 3.8 ± 1.5 × 10⁻⁴ |
| | bIL170 | JH-22 | 3.1 ± 1.2 × 10⁻⁴ |
| c2 species^{a} | c2 | JH-20 | 5.2 ± 0.4 × 10⁻⁵ |
| | bIL67 | JH-20 | 2.0 ± 1.2 × 10⁻⁴ |
| | ml3 | JH-20 | 3.4 ± 0.3 × 10⁻⁴ |
| | eb1 | JH-20 | 2.2 ± 0.7 × 10⁻⁴ |
| P335 species^{b} | ul36 | JH-23 | 1.0 |
| | KITI | JH-23 | 1.6 |
| | Ø31 | JH-23 | 1.0 |
| | Ø50 | JH-23 | 1.0 |
| | Q33 | JH-23 | 0.7 |
| | Q30 | JH-23 | 0.8 |
| | P335 | JH-23 | 0.4 |

^{a}EOP of 936 and c2 species is 1.0 on both L. lactis subsp. cremoris MG1363 (MB112) and MB112 + pLC5 (JH-54). EOP of phages P008 and bIL170 is 1.0 on L. lactis subsp. lactis IL1403
^{b}EOP of P335 species is 1.0 on L. lactis subsp. cremoris (SMQ-86).

To test the φrm for efficiency against P335 phages, the φrm was inserted in a suitable host (SMQ-86) resulting in the strain JH-23. When tested against seven species of P335 phages EOP values around 1 were obtained. To rule out the possibility that modifications had taken place rendering the φrm inefficient, pJH2 was prepared from JH-23 and re-inserted into MB112 to give strain JH-26. Tests against phage p2 showed an intact φrm phenotype.

Those results showed that the φrm found on the chromosome of L. lactis subsp. cremoris MG1363 and expressed from pJH2 is effective against phages from most of the tested 936 species and all tested c2 species while no effect was seen on P335 species.

The results also showed that the φrm encoded by orf1 is efficient in both the subspecies (cremoris and lactis) of L. lactis.

Furthermore the results showed that EOP values did not vary whether the φrm was expressed from a promoter located in single copy on the chromosome of the host or from a strong promoter on the vector pJH2. This indicates that the efficiency of the system is not dependent on the copy number of the gene.

### Example 9

### Temperature Sensitivity

The efficiency of the φrm was tested against phage sk1 at 30° C. and 37° C. EOP values were in both cases around 10⁻⁴ (data not shown) indicating that the φrm is stable within this temperature range.

### Example 10

### Type of Phage Resistance Mechanism

A series of microbiological experiments were conducted to determine the type of φrm encoded by orf1.

An adsorption assay showed that the level of adsorption of phage p2 to cells with the expressed φrm was 95.9±10.6% compared to wt MG1363 (data not shown).

An assay was conducted where the φrm⁺ strains JH-32, JH-46, JH-47, JH-48 and control MB112 was infected with sk1 that had been fluorescently labeled with the DNA binding dye SYBR-Gold. Following infection the fluorescently labeled phage DNA could be visualized under an epifluorescence microscope. Immediately following phage infection (MOI=10) of wt strain MB112 a fluorescent halo of adsorbed phages was seen surrounding the host cells. Less than 10 min after infection the fluorescent signal on the cell surface was decayed and instead a very bright fluorescent signal was observed in the center of the cell, thus indicating that the phage DNA had been injected into the host cell (data not shown).

The same result was obtained with the strains having the φrm⁺ phenotype. This supports the data from the adsorption assay and also shows that the phage DNA is being injected in the φrm containing cells. These results indicate that the φrm is not an adsorption or injection blocking mechanism.

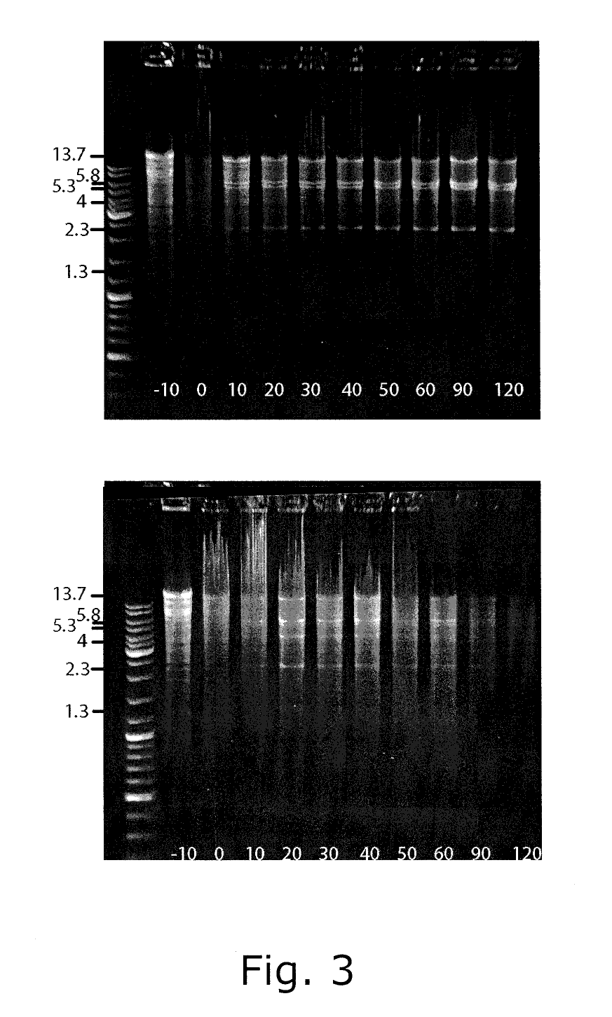

A cell survival assay showed no increased survival on cells harboring the φrm (Table 3) indicating that the host dies upon infection. The plaque size of phage p2 was smaller when assayed on φrm⁺ cells compared to φrm⁻ cells (Table 3). Finally, total DNA extraction from φrm⁺ cells during a time course experiment of infection with phage p2 showed phage DNA replication which persisted in the cell throughout the experiment (FIG. 3).

TABLE 3

| Assay | MB112 (wt) | JH-20 (abiV) |
|---|---|---|
| EOP^{a} | 1.0 | 4.8 ± 1.8 × 10⁻⁴ |
| ECOI (%)^{b} | 1.0 | 0.5 ± 0.2 |
| Burst size (pfu/cell)^{c} | 38.8 ± 5.7 | 11.1 ± 5.2 |
| fraction surviving cells^{d} | 6.1 ± 1.3 × 10⁻⁵ | 3.1 ± 0.3 × 10⁻⁶ |
| phage DNA replication^{e} | + | + (concatemeric) |
| plaque size (mm) | 1.5-1.7 | pinpoint - 0.7 |

^{a}n = 3
^{b}MOI = 0.5, n = 3
^{c}MOI = 0.2, n = 3
^{d}MOI = 5, n = 3
^{e}MOI = 2, n = 1

All the above results confirm that the mechanism is a φrm that functions as an abortive infection mechanism. This was named AbiV.

### Example 11

### Sequence Analysis of the 1.3 Kb DNA Fragment Containing the φrm

The DNA fragment cloned in pJH2 was sequenced (SEQ ID NO 3 and 5). The fragment consists of 1300 nucleotides. Nucleotides 1 to 1300 correspond to nucleotides 1021 to 2320 in the Lin sequence (GenBank acc.nr: AF324839). One significant open reading frame (orf) was found encoding the polypeptide sequence shown in SEQ ID NO 1. This gene encoding the φrm was named abiV (SEQ ID NO 3) and the translated protein was named AbiV (SEQ ID NO 1). The G+C content of the gene was found to be 31.7%. This value is typical for abi mechanisms which are known to have lower G+C contents compared to the normal 37% in L. lactis. Searches for promoter sequences upstream of abiV (bp 1 to 430) were performed but no suitable promoter could be found in this region. This corresponds well with the hypothesis of the φrm being silent in the wt strain MB112. The translation start codon was preceded (8 bp upstream) by a ribosome binding site (5′-TGAACGGAGAG-3′, underlined sequence matches consensus sequence).

### Example 12

### Analysis of the abiV Protein Encoded by abiV in pJH2

Since the abiV gene is the only orf in the cloned sequence of pJH2 and a frame shift mutation in this orf causes the phage sensitive phenotype, it is concluded that the protein encoded by this gene is responsible for the φrm⁺ phenotype. AbiV consists of 201 amino acids and has a molecular weight of 22692 Da. The pI was estimated to be 5.37.

The protein does not contain any putative transmembrane or signalpeptide motifs and it is therefore likely that the protein is cytosolic. Homology searches in databases did not reveal any homology (at amino acid or nucleotide level) to other lactococcal proteins or any proteins with known function. Likewise, no conserved domains were found in the protein.

The deduced function of AbiV is therefore new and the φrm is a novel abi mechanism.

### Example 13

### Effect of abiV on Phage Life Cycle

The effects of the AbiV system was tested on the phage p2 life cycle using the phage sensitive strain MB112 and the corresponding AbiV containing strain JH-20. The following results are summarized in table 3.

The propagation of p2 on JH-20 was inhibited as seen by the EOP of ca 10⁻⁴ and the plaque size was reduced from about 1.5 mm to <1 mm. Very few of the infected cells harboring the φrm survived infection.

The ECOI on JH-20 was 0.5±0.2% indicating that only 5 out of 1000 infected cells managed to release at least one viable phage. In these successful infections the burst size was reduced by 72% (from 38.8±5.7 in MB112 to 11.1±5.2 in JH-20).

The combined negative effects of AbiV on cell survival, ECOI and burst size were the cause of the reduced plaque size and EOP of p2 on JH-20.

The replication of phage DNA was followed in a 2 h phage infection experiment of p2 on phage resistant JH-20 and phage sensitive MB112 (FIG. 3). Phage DNA was visualized by digesting the total DNA prepared from an infected cell culture with EcoRV and comparing the resulting fragments run on an agarose gel with the EcoRV restriction map of phage p2.

Ten minutes after infection replication of phage DNA was observed in both strains. In MB112 the concentration of phage DNA decreases around 40 min after infection coinciding with lysis of the sensitive host culture. On the contrary, in JH-20 the phage DNA persists throughout the experiment which was terminated after 2 h. Inspecting the EcoRV digested phage DNA fragments, two bands of 1.3 and 4 kb respectively and a 5.3 kb fragment are seen in the phage sensitive culture. The 5.3 kb fragment is spanning the cos site on the phage DNA which is the site where the replicated phage DNA is cut into identical units of complete phage genomes before packaging of the DNA into the capsids. Therefore the 1.3 and 4 kb fragments represent DNA that has been cut at the cos site. The presence of both non-resolved and resolved DNA in the phage sensitive strain is due to the continuous DNA replication throughout the phage life cycle and the simultaneous packaging of already resolved DNA into the phage capsids. In JH-20 (AbiV⁺) only the 5.3 kb fragment is observed which indicates that the phage DNA is not cut at the cos site in this strain.

The above results show that AbiV works after phage DNA replication and is thus categorized as a late abi mechanism. The presence of concatemeric DNA fragments (cos site not cut) further suggests that the φrm might work at a late stage for example during packaging of phage DNA into the capsids.

### Example 14

### Phage Genes Involved in Sensitivity to abiV

A number of phage mutants capable of overcoming AbiV were isolated. On JH-20 AbiV-insensitive mutants of p2, sk1, jj50 and c2 were isolated and named p2.1, sk1.1, jj50.1 and c2.1, respectively. On JH-22, mutants of P008 and bIL170 were isolated and named P008.1 and bIL170.1, respectively.

The full genome of mutant p2.1 was sequenced revealing only mutations in the region around the early gene orf26 (SEQ ID NO 4). SEQ ID NO 4 encodes a polypeptide sequence denoted SEQ ID NO 2. The following polynucleotide mutations were found in phage p2.1 that escaped the AbiV-mechanism:
Two point mutations in orf26 leading to amino acid changes.
One point mutation in the intergenic region between orf26 and the upstream gene orf27
A 55 bp deletion including the startcodon and 6 downstream base pairs of orf26.

The homologues of p2 orf26 in the other phage mutants were sequenced. Nonsense mutations were observed in: orf26 (sk1.1), orf25 (jj50.1), orf33 (P008.1), e24 (bIL170.1) and a point mutation leading to an amino acid change (T to P) was seen in ell (c2.1).

These data show that AbiV-resistant phage mutants apparently fail to produce functional protein encoded by an early gene homologous to phage p2 orf26. In at least one phage mutant (p2.1), orf26 is the only gene which is mutated. Finally, phage 712 (936 species) is the only phage among the tested phages from the 936 and c2 species that does not contain an orf26 homologue. Among the wt phages of the 936 and c2 species, this phage is also the only one which is not sensitive to AbiV.

Based on the above results, it is concluded that a functional copy of phage p2 orf26 (and homologues in other phage species) is mandatory for successful φrm⁺ phenotype of AbiV. The gene is named say (sensitivity to abiV) and the translated putative protein was named Say. It is thus possible to strengthen the AbiV-mechanism by supplying the AbiV host cell with a polynucleotide sequence encoding wt Say.

A nucleotide blastn analysis orf phage p2 orf26 revealed a high degree of sequence homology to other lactococcal phage genes: jj50 orf25 (99.7%), sk1 orf26 (99.0%), P008 orf33 (91.4%), bIL170 e24 (90.6%). Furthermore the translated p2 orf26 showed a more distant relationship (29%) with phage c2 gene ell. Despite the low degree of homology the ell gene of phage c2 is involved in sensitivity to AbiV since a mutation in this gene helps the phage c2.1 escape AbiV. Therefore, sequences of either phage 936-like orf26 homologues (SEQ ID NO 2) or c2-like ell homologues (SEQ ID NO 7; DNA sequence: SEQ ID NO 8, derived from accession number NC001706 disclosing the complete genome of Lactococcus lactis phage c2), or variants or fragments thereof are a part of the present invention.

### Example 15

### Analysis of the Phage p2 Gene orf26 (Sav) and the Putative Protein (Sav) Encoded by this Gene

The DNA fragment containing phage p2 gene orf26 and the upstream intergenic region to orf27 was sequenced on both strands. The sequenced fragment contains 499 nucleotides (SEQ ID NO 5). The sav gene consists of 384 bp (SEQ ID NO 4). Upstream of say in a suitable (8 bp) distance is found a RBS sequence (GGATTGGGGGT, underlined sequence matches consensus sequence). No promoter sequence is found in the region between orf27 and say. This corresponds well with the genetic structure of this region in p2 and in the closely related phage sk1. In both phages orf26 is the last gene in a putative operon consisting of orf30 to orf26 where the promoter is upstream of orf30 (4).

The sav gene is located at the end of the early transcribed region of phage p2. The putative protein Say (SEQ ID NO 2) encoded by the gene say consists of 128 amino acids. It has a theoretical molecular weight of 15.3 kDa and an estimated pI of 4.62. Homology searches revealed homology to a number of putative proteins in related phages of the 936 and c2 species. However, no homology was found to proteins with known function. Nor was found any conserved domains in the protein. The protein is thus new and it has not previously been associated with sensitivity to phage resistance mechanisms. SEQ ID NO 7 is present in the database under accession number NC001706 and it has not previously been associated with sensitivity to phage resistance mechanisms.

The interaction of Say with AbiV is not known but the insensitivity to AbiV of phages with a deleted sav gene clearly indicates that say is involved in sensitivity of the phage to the φrm.

Co-expression of abiV and say in host cells will most likely enhance the efficiency of AbiV since the escaping mutant phages will have to mutate in other genes than say. Co-expression might also broaden the range of phages against which AbiV is effective. These are so far only hypotheses but they are in the process of being tested experimentally.

Since say has not previously been associated with any φrm, the AbiV φrm in the present invention is a new abi mechanism interacting in a so far unknown way with the sensitive phage. AbiV is therefore likely to be an efficient φrm capable of supplementing already isolated and used phage resistance mechanisms thus improving the field of phage resistance mechanisms.

The discovery of a phage gene involved in sensitivity to the Abi-resistance mechanism may be used for obtaining a phage resistance mechanism that is more efficient than use of the AbiV-mechanism alone. It is thus likely that the use of the wild type orf26-sequence encoding the polypeptide according to SEQ ID NO 2 and/or SEQ ID NO 7 will fully or partly prevent that the phage can escape the Abi-mechanism according to the present invention by supplying AbiV-sensitive protein (SaV) together with AbiV-protein.

The present invention thus relates to the use of polynucleotide sequences encoding both SEQ ID NO 1 and SEQ ID NO 2 and/or SEQ ID NO 7 (or a variant thereof) within a Lactococcus cell in order to exploit the synergy that exists in this combination. Compared to other known Abi-systems, the combination of SEQ ID NO 1 and SEQ ID NO 2 and/or SEQ ID NO 7 (or a variant thereof) in the same cell provides for a phage resistance mechanism that is extraordinarily efficient in preventing phage infections and thus preventing the emergence of AbiV-resistant phages.

### Example 16

### Use of Bacteria According to the Invention

The φrm according to the present invention can be used in connection with dairy starter cultures in existing dairy production plants to produce any fermented dairy food product.

### Example 17

### Construction of Expression Vector pLC5

The pGKV259 vector (18) was used as the starting molecule from which pLC5 was derived. pGKV259 was digested with PstI (located downstream from the P59 promoter) followed by gel purification. Two complementary oligonucleotides (5′-TGGATCCAAAGGAGGTCCTGCA-3′ and 5′-GGACCTCCTTTGGATCCATGCA-3′) were annealed together using standard procedures (16) to create a double stranded linker with PstI-compatible sticky ends. This linker also contained a unique BamHI site and a ribosome binding site (RBS: 5′-AGGAGG-3′). The linker was inserted into the PstI site of pGKV259 and the ligation mixture was transformed into E. coli MC1061. Transformants were selected on LB plates containing 20 μg/ml chloramphenicol. Positive clones with the linker inserted in the right direction were identified by colony PCR. Correct clones were later confirmed by sequencing.

Upon introduction of the linker into pGKV259, the PstI site on the 5′-side of the linker was disrupted whereas the one on the 3′-side was conserved. Thus, a unique PstI site was created 8-bp downstream from the RBS. Cloning of an insert harboring its own ATG start codon into the PstI site of pLC5 enables efficient transcription from the P59 promoter, and translation from the introduced RBS. For this invention, however, the native RBS of AbiV and not the RBS in the vector was used for translation of the protein.

### Example 18

### RNA Isolation, Purification and RT-PCR Analysis of Transcription

Overnight cultures were diluted 100-fold and grown to OD₆₀₀=0.5 at 37° C. Aliquots (2 ml) were harvested by quick centrifugation (20,000 g, 30 sec) and the pellet was resuspended in a solution of 0.5 M sucrose with 60 mg/ml lysozyme. Following incubation (37° C., 15 min), the cells were pelleted and resuspended in 1 ml TRIzol Reagent (Invitrogen). Total RNA was isolated according to the manufacturer's instructions. Prior to reverse transcription (RT)-PCR, the RNA samples were treated with the DNase based TURBO DNA-free kit (Applied Biosystems).

RT-PCR was carried out using the RevertAid First Strand cDNA Synthesis kit (Fermentas) as recommended by the manufacturer. As a control, the RT-PCR procedure was carried out without reverse transcriptase to ensure that the RNA samples were free of contaminating DNA.

### Example 19

### Activation: Mutants of L. Lactis Subsp. cremoris MB112 Spontaneously Expressing AbiV

Cultures of L. lactis MB112 in exponential growth were mixed with the lytic phage sk1 (MOI>1) in presence of 10 mM CaCl₂ and incubated 10 min at room temperature before plating and incubation at 36° C. overnight. Spontaneous mutants were observed with a frequency of ca. 10⁻⁸. Forty single colonies were purified and cross-streaked against phages sk1, p2, 712 and p2.1. A bacterial mutant expressing AbiV is expected to be resistant to sk1 and p2 but sensitive to 712 and p2.1 (Table 1 and Table 2). Possible candidates were tested with EOP for resistance to phages p2, 712, p2.1. One mutant (JH-80) revealed the expected pattern of a mutant expressing AbiV with EOP values of 2×10⁻⁵, 0.75 and 0.8, respectively.

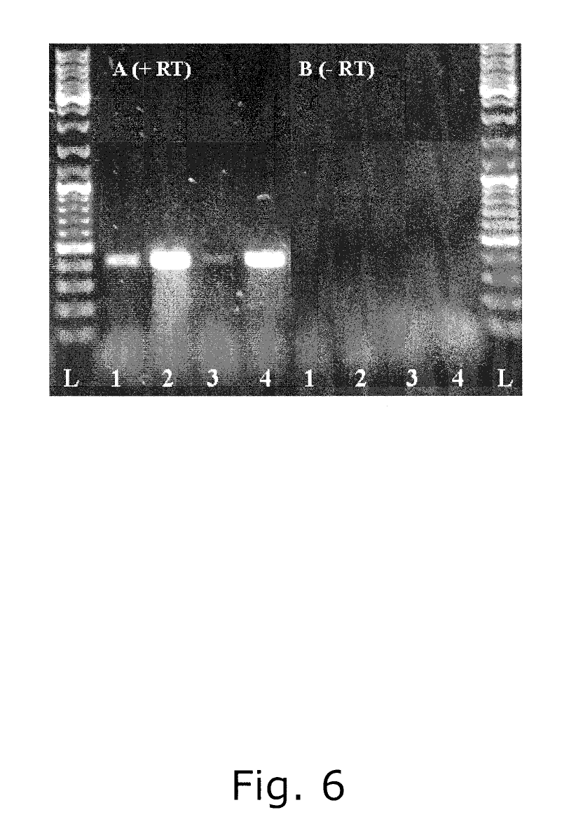

This mutant was investigated for transcription of the abiV gene using reverse transcriptase PCR(RT-PCR) (FIG. 6), as described in example 18.

These results demonstrate that it is possible to obtain mutants of L. lactis strains carrying abiV on the chromosome which spontaneously express AbiV. This experiment demonstrates that it is possible to obtain phage resistant bacteria expressing AbiV without using genetic modification. This is particular interesting for the dairy industry that prefers to avoid the use of genetically modified organisms (GMO). Furthermore the method should be applicable to non-expressed φrms in general which are located on bacterial genomes.

### Example 20

### Conjugal Transfer of abiV

In order to improve the non-GMO alternative of the φrm invention a conjugation experiment was conducted in which the φrm was transferred from the chromosome of JH-32 (donor) to MG1614 (recipient) (Table 4). Briefly, donor and recipient were recovered from plates and mixed at high cell densities (OD₆₀₀=40). After 2 min incubation the cell mixture was plated on non-selective plates and incubated overnight in anaerobic conditions. The cells were then recovered from the plates and plated with selection for donor (erythromycin resistance), recipient (rifampicin resistance) and transconjugants (erythromycin and rifampicin resistance), respectively.

Since the erythromycin resistance gene is inserted just upstream of abiV in JH-32 the erythromycin resistance phenotype was used to select for transfer of this gene to MG1614 hoping that abiV would be transferred along with it. Rifampicin resistance was used to select for MG1614.

A number of transconjugant candidates were isolated and purified. The additional phenotypes (resistance to phage, streptomycin and fluorouracil) were used to test the isolated candidates for verification of the phage+erythromycin resistance from JH-32 to MG1614. In JH-81 the expected pattern was observed (Table 4). An EOP value of 10⁻⁴ which is similar to other EOP values obtained with AbiV (Table 2) makes it plausible that abiV was transferred and expressed in MG1614.

This experiment demonstrated that it is possible to transfer AbiV by conjugation from one bacterium to another. Conjugation is not considered as genetic modification and the method is thus suitable for the industry for transferring AbiV between bacterial strains in a non-GMO manner.

TABLE 4
Phenotype of donor (D), recipient (R) and transconjugant (T). Selection for
transconjugants was done using erythromycin and rifampicin.

| Resistance | Phage | Erythromycin | Rifampicin | Streptomycin | Fluorouracil |
|---|---|---|---|---|---|
| JH-32 (D) | yes | yes | no | no | yes |
| MG1614 (R) | no | no | yes | yes | no |
| JH-81 (T) | yes | yes | yes | yes | no |

### Example 21

AbiV is a chromosomally-encoded phage resistance mechanism that is silent in the wild-type phage sensitive strain Lactococcus lactis subsp. cremoris MG1363. Spontaneous phage resistant mutants of L. lactis MG1363 were analyzed by reverse transcriptase PCR and shown to express AbiV, which was likely related to point mutations in the upstream region. Conjugal transfer of abiV was also demonstrated between two lactococcal strains. To the knowledge of the inventor, this is the first report of conjugal transfer of a chromosomally-encoded phage resistance mechanism.

Industrial milk fermentation is often dependent on the well characterized metabolic features of commercial starter cultures, which contain strains of lactic acid bacteria. However, milk fermentation failure due to dairy-adapted virulent phages that are infecting these specialized bacterial cultures is a persistent problem for the dairy industry. Decades of research has led to the discovery of a number of defense systems in Lactococcus lactis cells including inhibition of phage adsorption and DNA entry, restriction/modification (R/M) systems, and abortive infection (Abi) mechanisms. These lactococcal mechanisms have been used extensively in a relatively small number of industrial strains, which has favored the emergence of specific phage mutants that are insensitive to the natural anti-phage barriers. This viral evolutionary process has led to a lasting search for new ways to protect cultures against phage infection. Moreover, to avoid the use of the genetic engineering technology, the dairy industry is currently depending on the isolation of novel natural phage resistance barriers in a given wild-type L. lactis isolate that can be naturally transferred into industrial starter strains.

Many lactococcal R/M and Abi systems are plasmid encoded and some of them can be easily transferred from one strain to another through conjugation. This genetic transformation process is universally accepted and has been successfully utilized to create phage resistant starter cultures. Some phage resistance mechanisms are also chromosomally encoded. However, their industrial application is rather limited because they cannot be transferred into the desired industrial strains without the use of genetic engineering. Recently, we isolated a novel chromosomally-encoded Abi mechanism named AbiV that is active against lactococcal phages 17 (936 and c2 species). AbiV is silent in the phage sensitive strain L. lactis subsp. cremoris MG1363 but it can be activated when a promoter is provided. Here, we report the isolation of natural bacteriophage-insensitive mutants (BIMs) of L. lactis MG1363 that spontaneously express AbiV. Furthermore, the inventors demonstrate that abiV can be transferred to lactococcal strains by conjugation.

Isolation of mutants of L. lactis MB112 spontaneously expressing AbiV. To investigate if MG1363 could mutate spontaneously to express AbiV, the inventors isolated mutants that could grow in the presence of the virulent phage sk1. Ten independent cultures of L. lactis MB112 (L. lactis MG1363, Δupp) were exponentially grown at 30° C. in M17 medium supplemented with 0.5% glucose and then mixed with phage sk1 (MOI>1) in presence of 10 mM CaCl2. The phage-infected bacterial culture was then incubated 10 min at room temperature before plating and incubating overnight at 36° C. BIMs that spontaneously gained resistance to sk1 were observed at a frequency of 10-8. Fifty-six colonies were picked randomly among the ten independent cultures, purified, and cross-streaked against virulent phages sk1, p2, 712 and the AbiV-insensitive mutant p2.1. A BIM expressing AbiV is expected to be resistant to phages sk1 and p2 but sensitive to 712 and p2.1 due to the absence of a functional AbiV-target gene (say) in the two latter phages. Of the 56 BIMs mutants, eight of them had the expected efficiency of plaquing (EOP) values. One BIM (L. lactis JH-80) was selected for further analyses and the EOP values were 2×10⁻⁵ (phage p2), 0.75 (phage 712), and 0.8 (phage p2.1), suggesting that L. lactis JH-80 may now be expressing AbiV.

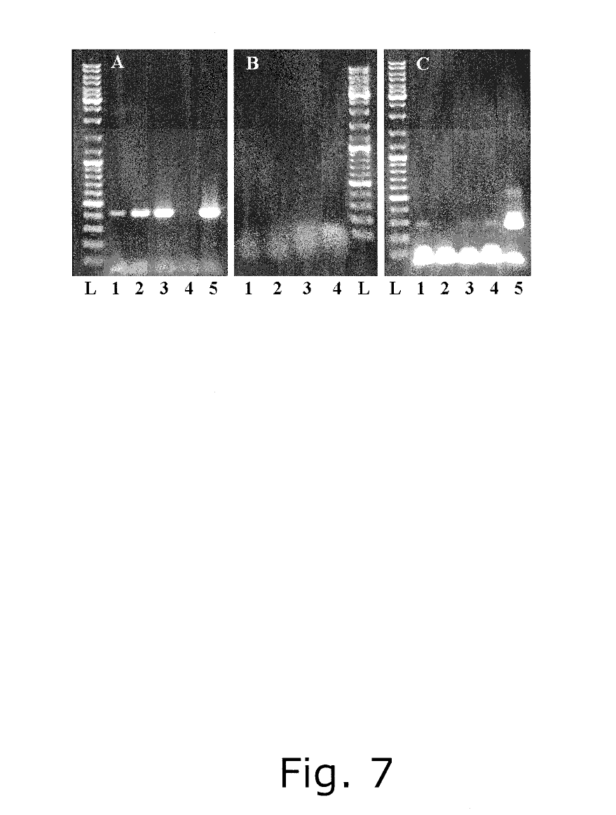

To verify that the phage resistance phenotype was indeed caused by the production of AbiV, the transcription of abiV in L. lactis JH-80 was investigated using reverse transcriptase PCR (RT-PCR) as described previously. The RT-PCR was performed on RNA isolated from L. lactis JH-80, but also on L. lactis JH-20 (L. lactis MB112 containing abiV cloned into the expression vector pLC5), JH-54 (L. lactis MB112 containing only the expression vector pLC5 without abiV) and JH-32 (L. lactis MB112 expressing abiV due to the integration of pGhost9::ISS1). While levels of abiV mRNA in L. lactis strains JH-20 and JH-32 were the highest, JH-80 also showed transcription as compared to no transcription in L. lactis JH-54 (FIG. 7A). No PCR products were obtained in control experiments (by omitting the RT enzyme) indicating that the RNA preparations were free of contaminating DNA (FIG. 7B). The above data demonstrates that L. lactis BIMs can be isolated, which spontaneously express AbiV, thereby conferring phage resistance to the cell without genetic modifications.

In an attempt to elucidate the mutation(s) in the BIM L. lactis JH-80 (AbiV+), the inventors PCR-amplified a 1300-bp region (nucleotides 1828 to 3140 in GenBank AF324839) which included abiV and the upstream gene trans. The size of the DNA fragment was the same for L. lactis JH-80 and MG1363, indicating that no DNA deletions or major rearrangements had occurred (results not shown). The PCR-amplified DNA fragments were sequenced and 6 point mutations were found in L. lactis JH-80. Three point mutations were approximately 1 kbp upstream of abiV while the three other point mutations were about 400 bp upstream of abiV, No lactococcal consensus promoter was observed in these regions. All mutations were within the upstream gene trans encoding a putative transposase. No terminator structure exists between abiV and trans leaving the possibility that promoter activity upstream of trans caused the increased expression of abiV. The inventors therefore determined the transcription level of trans in L. lactis JH-80, JH-32, JH-20, and JH-54. The inventors observed faint bands, indicating that trans was transcribed at low levels in all strains (FIG. 7C). However, nine other transposases that share 99% nucleotide similarity with trans exist on the genome of L. lactis MG1363, thereby complicating the analysis of trans expression. Nevertheless, since the transcription levels of trans were similar and low in all the tested strains, the inventors concluded that the increased promoter activity upstream of trans was not the cause of the elevated levels of abiV in JH-80. Therefore, it is likely that the weak promoter activity is due to the mutations within trans. It should be noted that some of the mutations in trans were silent while others caused amino acid changes (Q-29-R, A-216-P, and R-227-G). Therefore, it is still possible that an altered trans gene product was somehow involved in creating the increased level of abiV transcription.

Transfer of abiV by chromosomal conjugation. Most known Abi mechanisms are plasmid encoded, though it has been argued that this overrepresentation could be due to the technical advantages of isolating the plasmid encoded Abi systems. AbiV is located on the chromosome of L. lactis MG1363 and possibly not readily transferable to another strain, as compared to plasmid-encoded systems. However, conjugation of chromosomal genetic material has been previously observed in L. lactis, which is facilitated by the chromosomally encoded sex-factor (cluA) present, among others, in L. lactis MG1363. This genetic element permits the exchange of genetic material between lactococcal strains by chromosomal transfer and subsequent recombination. Therefore, the inventors investigated whether the inventors could take advantage of this ability to transfer an active AbiV from one strain to another.

As proof of concept, the inventors used the donor strain L. lactis JH-32 (AbiV+, erythromycin resistant (ErmR), fluorouracil resistant (FUR)) and the recipient strain L. lactis MG1614, a MG1363 derivative that is resistant to rifampicin (RifR) and streptomycin (StrR). In L. lactis JH-32, AbiV is activated by the vector pGhost9::ISS1 inserted immediately upstream of abiV on the bacterial chromosome. Briefly, donor and recipient cells were grown separately on GM17 plates and subsequently recovered with saline (0.9% NaCl) and then mixed at ratios of 1:1, 1:3, and 1:9. The mixtures were immediately plated (0.1 ml plate-1) on GM17 and incubated in anaerobic jars overnight at 36° C. This incubation temperature was selected to avoid excision of the integrated pGhost9::ISS1 in L. lactis JH-32. Cells were recovered from GM17 plates with saline and incubated again anaerobically (48 h, 30° C.) but on GM17 plates containing erythromycin (3 μg ml-1) and rifampicin (100 μg ml-1). These two selection markers were used to select for L. lactis MG1614 (rifampicin resistant) transconjugants that have acquired pGhost9::ISS1 (erythromycin resistance). The inventors envisioned that if pGhost9::ISS1 was successfully transferred to L. lactis MG1614 by conjugation, abiV would most likely be as well, due to their close location on the chromosome of L. lactis JH-32.

The lactococcal colonies that grew on GM17 plates containing erythromycin and rifampicin were then tested for their sensitivity to fluorouracil (0.3 μg ml-1) and their resistance to streptomycin (200 μg ml-1) and phages. By using this phage- and streptomycin-free selection approach, the inventors virtually eliminated the risk of isolating false positives due to spontaneous mutations causing the resistance phenotype. Transconjugants with the phenotype, RifR, StrR, FUS, ErmR, and phageR are expected to be derivatives of MG1614 (RifR, StrR) that have acquired ErmR and phageR from JH-32 by chromosomal transfer.

Seven putative transconjugants (ErmR, RifR) were first isolated after two days of anaerobic incubation (36° C.). Five of these mutants were derived from the donor and had acquired a spontaneous RifR resistance. One mutant was derived from the recipient with a spontaneous ErmR mutation. However, one mutant (L. lactis JH-83) had the expected phenotype (FUS, ErmR, RifR, StrR, and phageR). In fact, phage p2 had an EOP of 10-4 on L. lactis JH-83. This data strongly suggests that L. lactis JH-83 is a transconjugant of L. lactis MG1614 that has acquired an activated AbiV by conjugation and recombination, the inventors performed a PCR amplification of the chromosomal region upstream of abiV. The patterns of amplified PCR fragments were identical for JH-32 and JH-83 while being different from the MG1614 pattern, which indicated that abiV and the ErmR had been successfully transferred from L. lactis JH-32 to L. lactis MG1614 (data not shown). Furthermore, the inventors sequenced the rpsL gene of the four strains L. lactis MB112, MG1614, JH-32, and JH-83 (data not shown). A specific K-R amino acid substitution in rpsL is known to cause streptomycin resistance in different bacterial species. The same mutation was found in L. lactis MG1614 and JH-83 while L. lactis MB112 and JH-32 had the wild type sequence. Since rpsL was identical in JH-83 and MG1614 and streptomycin was not used as selection marker, the inventors concluded that indeed MG1614 was the parental origin of JH-83. To our knowledge this is the first demonstration of a conjugal transfer of a chromosomally encoded phage resistance mechanism.

Taken altogether, the above data indicates that the chromosomally-encoded abiV can be spontaneously activated and also naturally transferred to other lactococcal strains by conjugation. This study suggests that the search for novel chromosomally-encoded Abi mechanism should be revisited and this may open up new ways to construct natural phage-resistant strains for large-scale industrial applications.

### REFERENCES IN TABLE 1

1. Bolotin, A., P. Wincker, S. Mauger, O. Jaillon, K. Malarme, J. Weissenbach, S. D. Ehrlich, and A. Sorokin. 2001. The complete genome sequence of the lactic acid bacterium Lactococcus lactis ssp lactis IL1403. Genome Research 11:731-753.
2. Emond, E., B. J. Holler, I. Boucher, P. A. Vandenbergh, E. R. Vedamuthu, J. K. Kondo, and S. Moineau. 1997. Phenotypic and genetic characterization of the bacteriophage abortive infection mechanism AbiK from Lactococcus lactis. Appl. Environ. Microbiol. 63:1274-1283.
3. Hayes, F., C. Daly, and G. F. Fitzgerald. 1990. Identification of the Minimal Replicon of Lactococcus lactis subsp. lactis UC317 Plasmid pCI305. Appl. Environ. Microbiol. 56:202-209.
4. Leenhouts, K., G. Buist, A. Bolhuis, B. A. ten, J. Kiel, I. Mierau, M. Dabrowska, G. Venema, and J. Kok. 1996. A general system for generating unlabelled gene replacements in bacterial chromosomes. Mol. Gen. Genet. 253:217-224.
5. Gasson, M. J. 1983. Plasmid complements of Streptococcus lactis NCDO 712 and other lactic streptococci after protoplast-induced curing. J. Bacteriol. 154:1-9.
6. Maguin, E., H. Prevost, S. D. Ehrlich, and A. Gruss. 1996. Efficient insertional mutagenesis in lactococci and other gram-positive bacteria. J. Bacteriol. 178:931-935.
7. Martinussen, J. and K. Hammer. 1994. Cloning and characterization of upp, a gene encoding uracil phosphoribosyltransferase from Lactococcus lactis. J. Bacteriol. 176:6457-6463.

### REFERENCE LIST

1. Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. Nucleic Acids Res. 25:3389-3402.
2. Behnke, D. and H. Malke. 1978. Bacteriophage interference in Streptococcus pyogenes. I. Characterization of prophage—host systems interfering with the virulent phage A25. Virology 85:118-128.
3. Bolotin, A., P. Wincker, S. Mauger, O. Jaillon, K. Malarme, J. Weissenbach, S. D. Ehrlich, and A. Sorokin. 2001. The complete genome sequence of the lactic acid bacterium Lactococcus lactis ssp lactis IL1403. Genome Research 11:731-753.
4. Chandry, P. S., B. E. Davidson, and A. J. Hillier. 1994. Temporal transcription map of the Lactococcus lactis bacteriophage sk1. Microbiology 140 (Pt 9):2251-2261.
5. Dupont, K., T. Janzen, F. K. Vogensen, J. Josephsen, and B. Stuer-Lauridsen. 2004. Identification of Lactococcus lactis genes required for bacteriophage adsorption. Appl. Environ. Microbiol. 70:5825-5832.
6. Emond, E., B. J. Holler, I. Boucher, P. A. Vandenbergh, E. R. Vedamuthu, J. K. Kondo, and S. Moineau. 1997. Phenotypic and genetic characterization of the bacteriophage abortive infection mechanism AbiK from Lactococcus lactis. Appl. Environ. Microbiol. 63:1274-1283.
7. Hayes, F., C. Daly, and G. F. Fitzgerald. 1990. Identification of the Minimal Replicon of Lactococcus lactis subsp. lactis UC317 Plasmid pCI305. Appl. Environ. Microbiol. 56:202-209.
8. Hill, C., I. J. Massey, and T. R. Klaenhammer. 1991. Rapid Method To Characterize Lactococcal Bacteriophage Genomes. Appl. Environ. Microbiol. 57:283-288.
9. Leenhouts, K., G. Buist, A. Bolhuis, B. A. ten, J. Kiel, I. Mierau, M. Dabrowska, G. Venema, and J. Kok. 1996. A general system for generating unlabelled gene replacements in bacterial chromosomes. Mol. Gen. Genet. 253:217-224.
10. Maguin, E., H. Prevost, S. D. Ehrlich, and A. Gruss. 1996. Efficient insertional mutagenesis in lactococci and other gram-positive bacteria. J. Bacteriol. 178:931-935.
11. Mahony, J., H. Deveau, G. S. Mc, M. Ventura, C. Canchaya, S. Moineau, G. F. Fitzgerald, and S. D. van. 2006. Sequence and comparative genomic analysis of lactococcal bacteriophages jj50, 712 and P008: evolutionary insights into the 936 phage species. FEMS Microbiol. Lett. 261:253-261.
12. Martinussen, J. and K. Hammer. 1994. Cloning and characterization of upp, a gene encoding uracil phosphoribosyltransferase from Lactococcus lactis. J. Bacteriol. 176:6457-6463.
13. Moineau, S., S. Pandian, and T. Klaenhammer. 1994. Evolution of a lytic bacteriophage via DNA acquisition from the Lactococcus lactis chromosome. Applied and Environmental Microbiology 60:1832-1841.
14. Moineau, S., E. Durmaz, S. Pandian, and T. R. Klaenhammer. 1993. Differentiation of Two Abortive Mechanisms by Using Monoclonal Antibodies Directed toward Lactococcal Bacteriophage Capsid Proteins. Appl. Environ. Microbiol. 59:208-212.

15. Noble, R. T. and J. A. Fuhrman. 2000. Rapid virus production and removal as measured with fluorescently labeled viruses as tracers. Appl. Environ. Microbiol. 66:3790-3797.
16. Sambrook, J. and D. W. Russell. 2001. Molecular cloning, a laboratory manual. Cold Spring Habour Laboratory Press, Cold Spring Habour, N.Y.
17. Sanders, M. E. and T. R. Klaenhammer. 1980. Restriction and Modification in Group N Streptococci: Effect of Heat on Development of Modified Lytic Bacteriophage. Appl. Environ. Microbiol. 40:500-506.
18. van der Vossen, J. M., D. van der Lelie, and G. Venema. 1987. Isolation and characterization of Streptococcus cremoris Wg2-specific promoters. Appl. Environ. Microbiol. 53:2452-2457.

1. A transconjugated lactic acid bacterium, wherein the transconjugated genetic material is chromosomal DNA encoding and expressing a genetic determinant for a phage resistance mechanism (φrm).

2-26. (canceled)

27. The transconjugated lactic acid bacterium according to claim 1, wherein said lactic acid bacteria is a Lactococcus transconjugant.

28. The transconjugated lactic acid bacterium according to claim 27, wherein said Lactococcus is Lactococcus Lactis.

29. The transconjugated lactic acid bacterium according to claim 1, wherein said phage resistance mechanism is an abortive infection mechanism (Abi).

30. The transconjugated lactic acid bacterium according to claim 29, wherein said abortive infection mechanism (Abi) is selected from the group consisting of AbiV, AbiA, AbiF, AbiK, AbiP, AbiR, AbiT, AbiC, AbiE, Abil, AbiQ, AbiB, AbiD1, AbiU, and AbiZ.

31. The transconjugated lactic acid bacterium according to claim 29, wherein said abortive infection mechanism (Abi) is AbiV.

32. The transconjugated lactic acid bacterium according to claim 1, wherein said genetic determinant for said phage resistance mechanism is encoded by a polynucleotide sequence that encodes a polypeptide with at least 70% identity with SEQ ID NO 1, or a fragment thereof, and wherein expression of said polynucleotide confers at least one phage resistance mechanism to a transconjugated lactic acid bacterium.

33. A starter culture composition comprising a transconjugated lactic acid bacterium according to claim 1.

34. A method for preparing a fermented food product, said method comprising adding to the raw material or semi-manufacture to be fermented a transconjugated lactic acid bacterium according claim 1.

35. The method of claim 34, where said transconjugated lactic acid bacterium is provided as a starter culture composition comprising said bacterium.

36. A food product comprising a transconjugated lactic acid bacterium according to claim 1.

37. The food product according to claim 36, wherein said food product is a dairy product.

38. A method of preparing a transconjugated lactic acid bacterium according to claim 1, said method comprising culturing a donor bacterium comprising chromosomal DNA encoding said genetic determinant for said phage resistance mechanism (φrm) and a recipient lactic acid bacterium in a medium suitable for transconjugation, and subsequently isolating a transconjugated lactic acid bacterium conferring said phage resistance.

39. The method according to claim 38, where chromosomal encoded phage resistance mechanism is an abortive infection mechanism (Abi).

40. The method according to claim 39, wherein said phage resistance mechanism is AbiV.

41. The method according to claim 38, wherein said genetic determinant for phage resistance is coupled to a selection marker used for selection of a transconjugated lactic acid bacterium.

42. The method according to claim 41, wherein said selection marker is the erythromycin resistance gene (Erm^{r}).

43. A transconjugated lactic acid bacterium obtained by a method according to claim 38.

44. The transconjugated lactic acid bacterium according to claim 43, wherein said lactic acid bacteria is a Lactococcus.

45. The transconjugated lactic acid bacterium according to claim 44, wherein said lactic acid bacteria is Lactococcus Lactis.


SEQUENCE LISTING (18 sequences)

SEQ ID NO: 1 - protein, 202 aa, Lactococcus lactis
  VARIANT (202)..(202): Xaa can be any naturally occurring amino acid
MFDKDNYALGKMKNTLNTKESKFSLKSTDDLNKCIDHISVLIKDAYLLYTNESFATSTFISITIIEEVGKTHIGMFISENKDIKRGKDPLRNHKSKHAFGSLPTIKMGGRLNKAIGDEMIDKIVEDAETGELISIRESSLYADIIDDILEVPSEKISKEQSRALLLYAIECFDDSLVGYTHHSFEVSETTDELFEKLANNKX

SEQ ID NO: 2 - protein, 128 aa, Lactococcus 1 bacteriophage
MNYGTNNHYANEYGMELNEYFKHHFNYEELAGWYTMQVLKYLVRAGKKEGESYDKDRNKALDYAGELANLSNENELTEYTTDDIMGFAQDIADDFKQWKDERNNFKSEFTKEEIKAIDERYLEFIEEV

SEQ ID NO: 3 - DNA, 603 bp, Lactococcus lactis
atgtttgataaagacaactatgcattaggaaaaatgaagaatacccttaataccaaagaaagtaagttttctctaaagtcaactgatgatcttaataaatgcatcgatcatatttcagtcttaataaaagatgcatatctgctttatacgaatgaatcatttgccacttctacattcatttcaataacaattattgaagaagttggtaaaactcatataggtatgtttatcagtgagaataaagatataaagcgtgggaaagaccctttgagaaatcataaatccaaacacgcttttggatctcttccaactataaaaatgggaggacgacttaataaggctattggagatgaaatgattgataaaatcgtcgaagatgccgaaactggtgaacttatttcaatacgggagtcatctttgtatgcagatattattgatgatattcttgaagtacctagtgaaaaaattagtaaagaacaaagtagagcattgctcctttatgcgatagaatgttttgatgacagtttagttggctatacacatcattcatttgaagtatcagagacaactgatgagttgtttgaaaagttagcaaacaataaa

SEQ ID NO: 4 - DNA, 384 bp, Lactococcus bacteriophage
atgaattatggtacaaataagcactatgccaatgaatacggtatggaacttaacgaatactttaaacatcattttagctatgaagagcttgcaggctggtatacaatgcaggtattaaagtatctagtgagagctggcaagaaagagggtgaaagctacgacaaagaccgtaacaaggctttagactatgcaggagaacttgctaacttaagtaacgagaatgagcttacagaatacactactgacgacattatgggctttgcacaagatatagctgatgatttcaaacaatggaaaggcgaaagaaataactttaaatcagagttcacgaaagaagagataaaagcgattgatgaaagatacttggaatttattgaagaggtc

SEQ ID NO: 5 - DNA, 565 bp, Lactococcus bacteriophage
aagatacagtaaaaactttaatgatagctgtaggtataggctttacacttatcgctatcacttggataggtataattgcaacgttgcttattacatggattgggggtaacatctaatgaattatggtacaaataagcactatgccaatgaatacggtatggaacttaacgaatactttaaacatcattttagctatgaagagcttgcaggctggtatacaatgcaggtattaaagtatctagtgagagctggcaagaaagagggtgaaagctacgacaaagaccgtaacaaggctttagactatgcaggagaacttgctaacttaagtaacgagaatgagcttacagaatacactactgacgacattatgggctttgcacaagatatagctgatgatttcaaacaatggaaaggcgaaagaaataactttaaatcagagttcacgaaagaagagataaaagcgattgatgaaagatacttggaatttattgaagaggtctaaagttaattcttgacaaatataaagtaatttgataatattgttttatagaaaggggattaaaca

SEQ ID NO: 6 - DNA, 1300 bp, Lactococcus bacteriophage
aaaaagagagtgggtgtatcaatttaaatataagaactttgaagaagcctatcagagtattttctggtacatcgaagccttttataattcaaaacgaatccatcaaagtttagggtatcttacacctaatcaatttgaaaaggtaagtgcttaaaataaatagattaaaattctacgtttgttactctaaaaacttgacttaacgtcacttcttgagttaacttcgcataataaagaaaacaaagactttttggatgaaatagacaaaacttactctaaaattgatactgtcaatactaaagttagacaaactgaagtagctgcaactactaatcaacttgcgctaactaaagcaaatgtacaaattcatacccttttagtaattgctagtaattattatcaatcagtatggatccagattaaagaatgaacggagagttttatgtttgataaagacaactatgcattaggaaaaatgaagaatacccttaataccaaagaaagtaagttttctctaaagtcaactgatgatcttaataaatgcatcgatcatatttcagtcttaataaaagatgcatatctgctttatacgaatgaatcatttgccacttctacattcatttcaataacaattattgaagaagttggtaaaactcatataggtatgtttatcagtgagaataaagatataaagcgtgggaaagaccctttgagaaatcataaatccaaacacgcttttggatctcttccaactataaaaatgggaggacgacttaataaggctattggagatgaaatgattgataaaatcgtcgaagatgccgaaactggtgaacttatttcaatacgggagtcatctttgtatgcagatattattgatgatattcttgaagtacctagtgaaaaaattagtaaagaacaaagtagagcattgctcctttatgcgatagaatgttttgatgacagtttagttggctatacacatcattcatttgaagtatcagagacaactgatgagttgtttgaaaagttagcaaacaataaatagttaaatcttgagtttgattttgctgaatattctgcatttatcgggcggaatgatgcccttagactttgcaacagaacctcgattttaattcgttcagaataggttatactagacaaaagatcggctcctaaaaatgggtttgtgataaacaccattttaaaggaagctggtcttttttgtccaaacactggtcagacaattttggggcctatgatatttggtgttgatagataaaattcatcaacactattc

SEQ ID NO: 7 - protein, 123 aa, Lactococcus bacteriophage c2
  VARIANT (123)..(123): Xaa can be any naturally occurring amino acid
MAQDYYANKYGIQLEEFLIWGSEWDLKFWKYNFTTGQGFALTNALKYTVRAGKKPDEPYEKDMGKYNDYIDMAVKMGFERSEAEDWVALQKSIFEEFKGKKAEIEELEKRKEMKENDEIRRLX

SEQ ID NO: 8 - DNA, 366 bp, Lactococcus bacteriophage c2
atggctcaagattattatgcaaataagtacggtattcaattagaagagttcctgatttggggttctgaatgggacttaaaattttggaagtataactttacaactggtcaaggttttgcactaactaacgctttaaagtacactgtaagggcagggaaaaagccagatgaaccgtatgaaaaagatatgggcaaatataacgactacatcgacatggctgttaaaatgggctttgaacggtctgaagcagaagactgggtagcacttcaaaaatcaatatttgaggagtttaaaggaaaaaaagcagaaattgaagaacttgaaaaaagaaaggaaatgaaagaaaatgatgaaattcgtcgcctt

SEQ ID NO: 9 - DNA, 11 bp, Lactococcus bacteriophage
agaaaggaggt

SEQ ID NO: 10 - DNA, 11 bp, Lactococcus bacteriophage
agaaaggaggt

SEQ ID NO: 11 - DNA, 17 bp, Lactococcus bacteriophage
gaagaaatggaacgctc

SEQ ID NO: 12 - DNA, 11 bp, Lactococcus bacteriophage
tgaacggagag

SEQ ID NO: 13 - DNA, 11 bp, Lactococcus bacteriophage
ggattgggggt

SEQ ID NO: 14 - DNA, 22 bp, Lactococcus bacteriophage
tggatccaaaggaggtcctgca

SEQ ID NO: 15 - DNA, 22 bp, Lactococcus bacteriophage
ggacctcctttggatccatgca

SEQ ID NO: 16 - DNA, 6 bp, Lactococcus bacteriophage
aggagg

SEQ ID NO: 17 - DNA, 1300 bp, Lactococcus lactis; identical to SEQ ID NO: 6

SEQ ID NO: 18 - DNA, 499 bp, Lactococcus bacteriophage
aagatacagtaaaaactttaatgatagctgtaggtataggctttacacttatcgctatcacttggataggtataattgcaacgttgcttattacatggattgggggtaacatctaatgaattatggtacaaataagcactatgccaatgaatacggtatggaacttaacgaatactttaaacatcattttagctatgaagagcttgcaggctggtatacaatgcaggtattaaagtatctagtgagagctggcaagaaagagggtgaaagctacgacaaagaccgtaacaaggctttagactatgcaggagaacttgctaacttaagtaacgagaatgagcttacagaatacactactgacgacattatgggctttgcacaagatatagctgatgatttcaaacaatggaaaggcgaaagaaataactttaaatcagagttcacgaaagaagagataaaagcgattgatgaaagatacttggaatttattgaagaggtc